(19) United States
(12) Patent Application Publication
(10) Pub. No.: US 2010/0249210 A1
(43) Pub. Date: Sep. 30, 2010

(54) METHODS AND COMPOSITIONS RELATED TO DLK-1 AND THE P38 MAPK PATHWAY IN NERVE REGENERATION

(76) Inventors: Michael Bastiani, Salt Lake City, UT (US); Erik Jorgensen, Salt Lake City, UT (US); Paola Nix, Salt Lake City, UT (US); Marc Hammarlund, Hamden, CT (US)

Correspondence Address: Ballard Spahr LLP, SUITE 1000, 999 PEACHTREE STREET, ATLANTA, GA, 30309-3915, US

(21) Appl. No.: 12/558,296

(22) Filed: Sep. 11, 2009

Related U.S. Application Data
(60) Provisional application No. 61/108,252, filed on Oct. 24, 2008.
(60) Provisional application No. 61/194,714, filed on Sep. 30, 2008.

(51) Int. Cl. A61K 31/7088; C12N 5/02; C12Q 1/68; C40B 30/04; C07H 21/02

(52) U.S. Cl. 514/44 A; 435/375; 435/6; 506/9; 536/24.5

(57) ABSTRACT

Disclosed are compositions and methods for treating neurodegenerative disease.

### CROSS-RELATED APPLICATIONS

This application claims the benefit of U.S. provisional patent application Ser. No. 61/108,252, filed Oct. 24, 2008, as well as U.S. provisional patent application 61/194,714, filed Sep. 11, 2008. The aforementioned applications are herein incorporated by this reference in their entirety.

### STATEMENT REGARDING FEDERALLY SPONSORED RESEARCH

This invention was made with government support under Grant 1R21 NS060275-01 awarded by the National Institutes of Health. The government has certain rights in the invention.

### BACKGROUND

A recent study from the American Spinal Injury Association reported that approximately 250,000 people are living with spinal cord injury in the United States and estimated a yearly cost of 22 billion dollars for their care. The need to stimulate neural outgrowth, which is also called axon regeneration, arises in the treatment of many diseases, including peripheral neuropathies (for example, diabetic or chemotherapy-induced), paralysis caused by spinal cord injury, motor neurone, disease, neurodegenerative diseases, for example, multiple sclerosis, Alzheimer's disease, and Parkinson's disease, and ischemia, caused for example by stroke.

Therapies to improve axon regeneration in human spinal cord injury are going to continue to fail until there is a comprehensive knowledge of the molecular signaling pathways that regulate axon regeneration.

What is needed in the art are methods and compositions that can be used to increase axon regeneration.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows dlk-1 is required for axon regeneration in β-spectrin mutants. (A) Cartoon showing development of axon morphology in control β-spectrin mutant (left) and in β-spectrin mutant lacking hypothetical regeneration gene (right). (B and C) GABA neurons in representative L4-stage β-spectrin mutants (unc-70) under control conditions or after dlk-1 RNAi. Scale bars: 20 μm. (D) High-magnification view of boxed region in (C). Arrows indicate inert axon stumps. Scale bar: 10 μm. (E and F) GABA neurons in representative L4-stage wild-type and dlk-1 mutant animals. Scale bars: 20 μm.

FIG. 2 shows dlk-1 is required in severed axons for growth cone initiation. (A) Regenerating axons 18-20 hours after laser surgery in a wild-type animal. Both severed axons have generated a growth cone (arrows), and the right axon has regenerated past the end of the distal fragment. Scale bar: 10 μm. (B) Axons in dlk-1 mutants fail to generate growth cones 18-24 hours after surgery. Scale bar: 10 μm. (C) DLK-1 acts cell intrinsically to mediate regeneration. (D) DLK-1 acts at the time of injury to mediate regeneration. Time of heat shock relative to surgery is indicated in column labels in hours. No heat shock is indicated by ‘no’. Bars in C and D show percentage of axons that initiated regeneration and 95% confidence interval (CI).

FIG. 3 shows dlk-1 controls growth cone initiation and morphology during axon regeneration. (A) Transient filopodium in a wild-type animal. Images were taken at 165 (left), 170 (center), and 180 (right) minutes after surgery. Scale bar: 5 μm. (B) Transient filopodium in a dlk-1 mutant animal. Images were taken at 475 (left), 480 (center), and 490 (right) minutes after surgery. Scale bar: 5 μm. (C) Representative axons in a wild-type animal 120 minutes after axotomy. Proximal and distal ends have retracted away from site of surgery, but proximal ends (arrows) show no evidence of regeneration. Scale bar: 10 μm. (D) A representative axon in an animal over expressing DLK-1 120 minutes after axotomy. The proximal end (arrow) has already regenerated past the retracted distal end. Scale bar: 10 μm. (E) Representative growth cones in a wild-type animal. Although these axons successfully initiated regeneration, the growth cones (arrows) have a dystrophic morphology. Scale bar: 10 μm. (F) Representative growth cones in an animal over expressing DLK-1 under the unc-47 promoter. These growth cones (arrows) have a compact morphology similar to growth cones observed during development. Scale bar: 10 μm. (G) Distribution of all times of filopodia initiation in wild type and dlk-1. Each dot represents a filopodium. (H) Time of first filopodium initiation in wild type and dlk-1. Bars show mean and standard error. (I) Rate of filopodia initiation in wild type and dlk-1. Bars show mean and standard error. (J) Time to initiate regeneration after surgery in wild type and dlk-1 over expressing (OE) animals. Initiation is defined as the appearance of the filopodia that becomes a growth cone. Each dot represents a single axon. (K) Percentage of regenerating axons that reached the dorsal cord after 18-24 hours. Error bars indicate 95% CI.

FIG. 4 shows MAP kinase signaling is required for axon regeneration. (A) Regeneration is eliminated by mutations in the DLK-1/MKK-4/PMK-3 MAP kinase module. (B) Other MAP kinase elements contribute to regeneration. (C) Activated DLK-1 has targets in addition to MKK-4 and PMK-3. (D) RPM-1 controls DLK-1 activity during axon regeneration. Bars in panels A-D show percentage of axons that initiated regeneration and 95% CI. (E) Model for function of MAP kinase signaling during axon regeneration.

FIG. 5 shows expression of p-unc-47::GFP in GABA motor neurons. (A) Micrograph of a L3 stage larvae expressing a p-unc-47::GFP N-terminal transcriptional fusion in all 26 GABA neurons. GFP is expressed throughout the cytoplasm and axonal process of these neurons, including the commissures and ventral and dorsal nerve cords. Scale 25 um. (B) Schematic of the GABA motor neurons expressing p-unc-47::GFP. The cell bodies and commissures of the thirteen post embryonic VD motor neurons are shown in green. The name and location of the commissures are on the dorsal side of the worm, the cell bodies, including the embryonic DD's and larval DVB, are on the ventral side of the worm. The gonad is yellow, the pharynx stippled in red.

FIG. 6 shows imaging single DD neuron from hatch to 96 hours illustrates the axon break and regeneration phenotype seen in the unc-70 genotype. Arrowheads point to growth cones, arrows to dorsal nerve cord process or degenerating fragments (see text for description).

FIG. 7 shows rescue of the unc-70 “break and regenerate phenotype (arrows A) by paralysis. Paralyzing unc-70 animals with RNAi targeting muscle myosin (unc-54) almost completely rescues the GABA nervous system (A vs. B). Quantified in C.

FIG. 8 shows RNAi screening control showing the knockdown of GFP expression (B. vs. A.) in worms after 9 days at 15 degrees C. eri-1; lin-15b strain increases sensitivity of neurons to RNAi. Each batch of approximately 50 genes to be screened includes this GFP RNAi control as well as a negative empty vector control.

FIG. 9 shows control L4 worm (unc-70; p-unc-17::spectrin; p-unc-17::mCherry; eri-1; lin-15b; p-unc-47::gfp). D axons that reach and extend in the dorsal nerve cord are scored in 10 worms/RNAi plate. In this example 8 axons were scored as successfully “regenerating” back to dorsal nerve cord. The average of hundreds of worms scored is 9-11 axons/worm in controls.

FIG. 10 shows the phenotype of dlk-1 RNAi. L4 worm (A.) scored for D commissures shows many “broken” axon stumps (B. arrowheads). No growth cones are apparent nor aberrant branches to indicate any regeneration had taken place.

FIG. 11 shows laser axotomy is used to validate the “strong” positives identified in the RNAi screen. The DD and VD motor neurons provide a well characterized model cellular system in which to characterize genes that function in regeneration. Laser cutting was done on the commissural axons of VD11, VD 10, DD5, and VD9 (circled in Top panel). These show robust regeneration and can be cut (red line) without affecting the growth or viability of L4 worms.

FIG. 12 shows laser axotomy of wild type D axons (A) and dlk-1 D axons (B). Wild type D neurons often regenerate growth cones within about 5 hours after axotomy dlk-1 D neuron axons have never been shown to regenerate a growth cone, even after 5 days.

FIG. 13 shows time lapse images dlk-1, wt, and Ex[dlk-1]. Note dlk-1 never extends a growth cone. Wt first extends a growth cone at 285 min. Ex[dlk-1] extends growth cone after only 10 min and reaches dorsal nerve cord by 285 min.

FIG. 14 shows spontaneous axon break and regeneration phenotype in unc-70 C. elegans. A. Intact DD axon and dorsal nerve cord branch. B. 24 hours later the same DD axon has broken and a regenerated growth cone (arrowhead) has extended from the proximal stump. The distal fragment is still visible in the dorsal nerve cord (arrow).

FIG. 15 shows axonal repair after laser “axotomy”. Both axons show gaps at arrowheads 20 min after laser axotomy. The left axon fully repairs by 210 minutes while the right axon still shows an obvious gap. By 350 min both axons look fully repaired. Images were recorded at 5 min. intervals. Scale 5

FIG. 16 shows regeneration in wild type C. elegans followed for 5 days. Each panel, pair represents regeneration of D neuron after laser axotomy. First picture is after 24 hrs and second is the same neuron after 5 days. If a neuron does not mount a good regeneration response in the first 24 hours after axotomy then it often fails to successfully regenerate to its target (dorsal nerve cord) even after 5 days. However, note that unsuccessful neurons do not exhibit degeneration or cell death.

FIG. 17 shows many amphid neurons do not regenerate their sensory dendrites. Over expression of candidate proteins in this neuron can restore its ability to regenerate.

FIG. 18 shows dlk-1 GABA nervous system is normal. Note continuous dorsal nerve cord (arrows) and normal D neuron commissural axons (example arrowheads).

FIG. 19 shows the unc-70; dlk-1 double mutant looks very similar to the dlk-1 RNAi phenotype identified in the screen of the “unc-70” screening strain (compare to FIG. 10). Note the four D commissural axons ending in stumps (arrowheads) and un-attached axon fragments (arrows). The dorsal nerve cord is completely absent, except for a small RME dorsal process extending from the nerve ring.

FIG. 20 shows wild type neurons often fail to regenerate fully back to the dorsal nerve cord, while neurons “over expressing” DLK-1 are almost always successful. Wild type neurons regenerate growth cones that often stall as hyper-branched structures with no well-defined growth cone (arrowheads). In contrast, the Ex[dlk-1] growth cones generally remain well defined with an obvious growth cone as they consistently advance to the dorsal nerve cord (arrowheads). The regenerating ex[dlk-1] growth cones maintain morphologies and dynamic behaviors much more similar to embryonic and post embryonic DD and VD growth cones (Knobel et al. 1999).

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such can, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Nucleic Acids

There are a variety of molecules disclosed herein that are nucleic acid based, including for example the nucleic acids that encode, for example, dlk-1 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### (a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., 1989).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### (b) Sequences

There are a variety of sequences related to, for example, dlk-1 as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

### (c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

### (d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of the disclosed nucleic acids or the genomic DNA or they can interact with the polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566; Forster and Altman, 1990).

Similarly, eukaryotic EGS/RNAse P-directed cleavage of RNA can be utilized to cleave desired targets within eukarotic cells. (Yuan et al. 1992; WO 93/22434; WO 95/24489; Yuan and Altman, 1995; Carrara et al. 1995). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

### 3. Peptides

### (a) Protein Variants

As discussed herein there are numerous variants of the proteins disclosed herein that are known and herein contemplated. In addition, to the known functional strain variants, such as those found in C. elegans, as well as those with homology from other species, there are derivatives of the proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants.

Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion.

Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof can be combined to arrive at a final construct. The mutations must not place the sequence out of reading frame and preferably will not create complementary regions that could produce secondary mRNA structure. Substitutional variants are those in which at least one residue has been removed and a different residue inserted in its place.

Substantial changes in function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also can be desirable. Deletions or substitutions of potential proteolysis sites, e.g., Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

Another way of calculating homology can be performed by published algorithms. Optimal alignment of sequences for comparison can be conducted by the local homology algorithm of Smith and Waterman, 1981, by the homology alignment algorithm of Needleman and Wunsch, 1970, by the search for similarity method of Pearson and Lipman, 1988, by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker 1989, Jaeger et al. 1989, and Jaeger et al. 1989, which are herein incorporated by reference for at least material related to nucleic acid alignment.

It is understood that the description of conservative mutations and homology can be combined together in any combination, such as embodiments that have at least 80% homology to a particular sequence wherein the variants are conservative mutations.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

All P values are two-tailed and are calculated against the wild type (EG1285 oxIs12, top row of this table) using Fisher's exact test (http://www.graphpad.com/quickcalcs/contingencyl.cfm).

Animals were maintained on NG agar plates with E. coli HB101 as a food source according to standard methods. See Table 1 for a listing of the strains and complete genotypes. The wild type was EG1285 oxIs12.

RNAi was performed by feeding as described (Kamath et al. 2003). Bleached embryos from MJB1046 basIs1; oxIs268, unc-70(s1502); eri-1 (mg366); lin-15(n765) gravid hermaphrodites were placed on dlk-1 RNAi bacteria. Plates were incubated at 15 C for 12-13 days. A minimum of 10 F1 L4 animals were scored by counting all GABA commissures contacting the dorsal cord. Because commissures are sometimes obscured by other processes, this method slightly underestimates the actual number. For example, 16 commissures were typically scored in wild-type animals, compared to the actual number of 19.

L4-stage hermaphrodites were mounted in 10 mM muscimol in M9 on an agarose pad under a coverslip. GFP expressing DD and VD motor neurons were imaged with a Microradiance 2000 confocal microscope using a Nikon 60×, 1.4 NA lens. Selected commissural axons were cut using a 440 nm MicroPoint Laser System from Photonic Instruments. After surgery, animals were recovered to an agar plate, and remounted for confocal imaging approximately 18-24 hours post-surgery. The imaged commissures were classified according to the following criteria: (1) regeneration (number of commissures with well-defined growth cones present on the proximal fragment and/or a net growth of 5 μm or more, (2) sprouting (number of commissures with small branches present on the proximal fragment), and (3) no regeneration (no change to proximal fragment after 18-24 hours). A minimum of 20 individuals (with 1-3 axotomized commissures each) were observed for most experiments.

In cases where the genetic background resulted in improved regeneration the data are likely to be an underestimate of successful regeneration. To ensure that only axons which had been completely severed were analyzed, experiments that did not include a growth cone and/or a recognizable distal fragment after 18-24 hours were eliminated. This approach underestimates successful regeneration because any experiment in which the regenerated axon obscured the distal stump appeared as an uncut commissure and would be eliminated. 95% confidence intervals were calculated by the modified Wald method, and two-tailed P values were calculated using Fisher's exact test (http://www.graphpad.com/quickcalcs/).

Molecular biology was done using standard techniques. PCR was done with Phusion DNA Polymerase (Finnzymes). Templates were either genomic DNA from mixed-stage N2 or first strand cDNA obtained by dT-primed reverse transcription of poly-A selected RNA from mixed-stage N2. Plasmids were assembled using multisite Gateway recombination (Invitrogen).

pPN12 (Punc-47:dlk-1 minigene):

A 5′ fragment (exon 1 and 2) was amplified from genomic DNA and a 3′ fragment (exons 3-11) from cDNA. The two fragments were ligated and a full-length product for generating a Gateway entry clone was obtained by amplifying with the following primers:

PN18
ggggacaagtttgtacaaaaaagcaggctggacatctaccacaatggtaa
cc
PN19
ggggaccactttgtacaagaaagctgggtgaattcggactgctccggcat
cg.

The final construct was obtained in a multisite Gateway reaction using the following constructs: pMH522 (Punc-47 [4-1]), pPN11 (dlk-1 minigene [1-2]), pMH473 (unc-54 terminator [2-3]), and pDEST [4-3].

pPN14 (Punc-47:dlk-1 cDNA-GFP):

The genomic portion of pPN11 was replaced with a BstEII-SalI digested cDNA fragment (PN16 acatctaccacaatggtaacc, PN31 cggagcttcttctggcattg). The final construct was obtained in a multisite Gateway reaction using the following constructs: pMH522 (Punc-47 [4-1]), pPN13 (dlk-1 cDNA [1-2]), pGH50 (GFP:unc-54 terminator [2-3]), and pDEST [4-3].

pMH524 (Phsp-16.2:dlk-1 cDNA-mCherry):

The final construct was obtained in a multisite Gateway reaction using the following constructs: pMH520 (Phsp-16.2 [4-1]), pPN13 (dlk-1 cDNA [1-2]), pGH38 (mCherry:unc-54 terminator [2-3]), and pCFJ150 MosSci [4-3].

Transgenic animals were obtained as described (Mello et al. 1991). MJB1011 was constructed by injecting EG4529 oxIs268 with pPN12 DNA at 30 ng/μL along with Pmyo-2:mCherry at 2 ng/μL as a co-injection marker. MJB1032 was constructed by injecting N2 with pPN14 DNA at 30 ng/μL along with Punc-47:mCherry at 20 ng/μl. EG5203 was constructed by injecting MJB1014 dlk-1(ju476); oxIs12 with Phsp-16-2::DLK-1-mCherry at 5 ng/μl together with Pmyo-2::GFP at 2 ng/μL as an injection marker and 1 kb ladder at 50 ng/μL as carrier. Stable transgenic lines were recovered based on GFP in pharynx muscles and subjected to heat shock. Two out of three lines tested showed weak mCherry in the intestine 24 hours after heat shock, showing that heat shock resulted in appreciable expression of the DLK-1-mCherry fusion protein. One of these lines was used for further experiments.

Mixed-stage animals were heat shocked at 33° C. for 1 hour on a sealed worm plate in a recirculating water bath. Animals containing the Phsp-16-2::DLK-1-mCherry array were selected based on pharynx GFP.

Confocal images collected at 5-minute intervals were analyzed for the appearance of filopodia (small, transient extensions from the axon tip or shaft). 11 axons in 8 animals were analyzed for wild type and 9 axons in 5 animals for dlk-1 (ju476). For wild-type axons, analysis was terminated at the appearance of the filopodium that eventually stabilized into a growth cone. The average time to this event was 460 minutes.

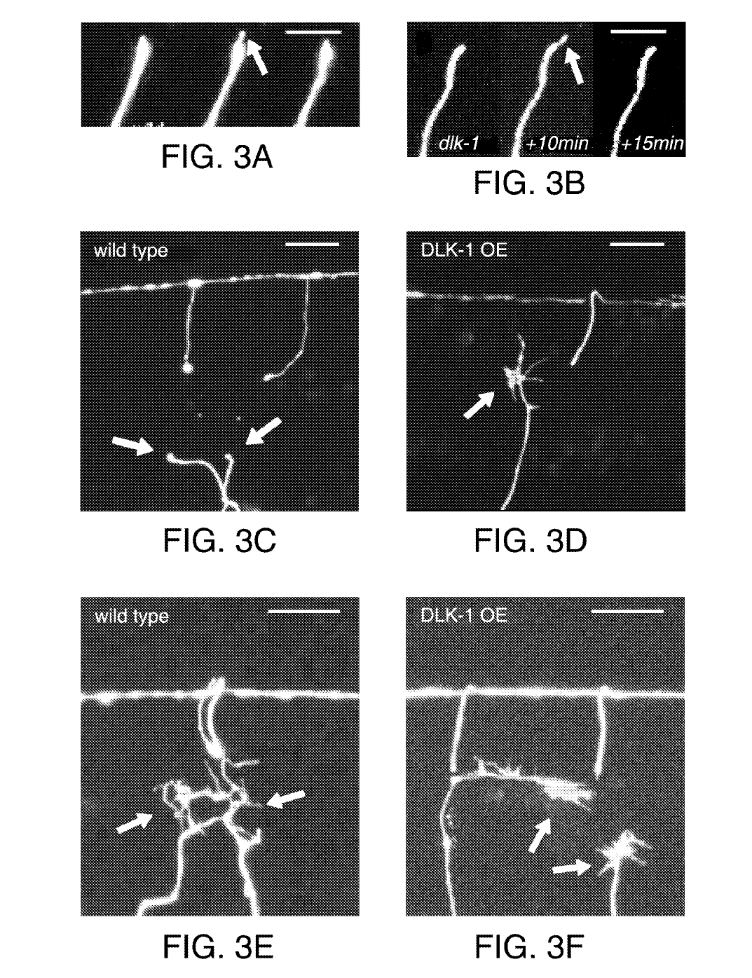

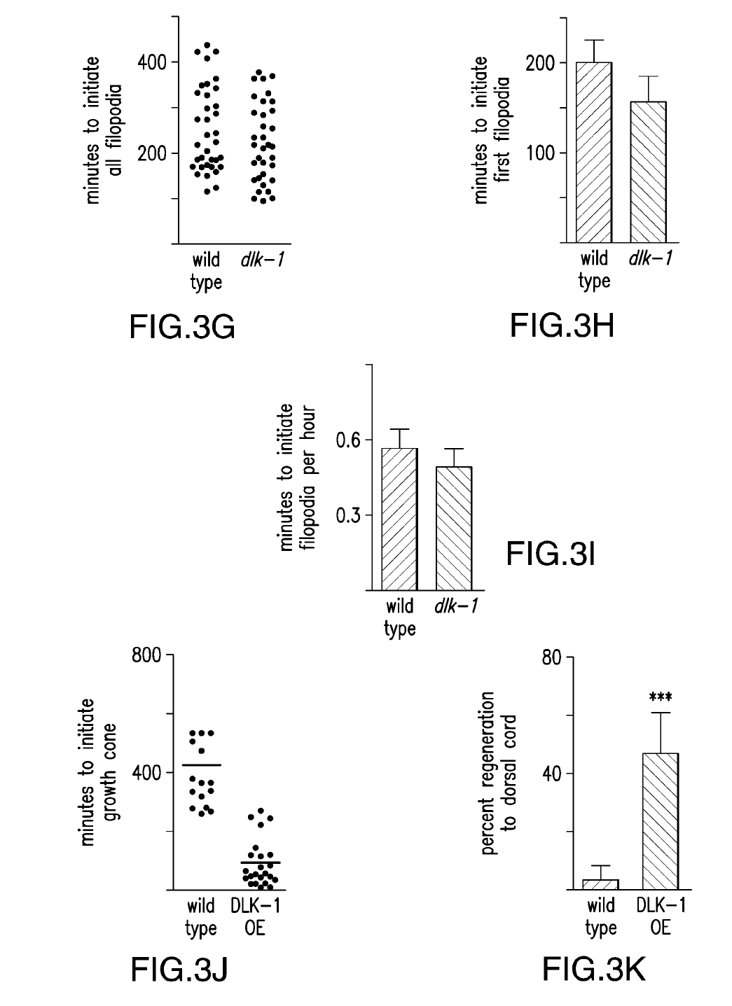

Since dlk-1 mutants never initiate growth cones, they continue to generate filopodia throughout the analysis, long after the average time to initiate a growth cone in wild type. Because these late events have no equivalent in wild-type animals, they were eliminated from the comparison in FIG. 3G. Only events in both genotypes occurring before 460 minutes were considered. FIG. 3H shows the average time and the standard error for the first event. FIG. 3I shows the average rate. For this analysis, the number of filopodia observed in each axon was divided by the time of observation for that axon. Analysis was truncated at growth cone initiation or 460 minutes, whichever came first.

Methods are similar to those described (Knobel et al. 1999) with the following changes. L4 worms were anesthetized with 1 μL of 10 mM muscimol (Sigma M1523) in M9 and mounted on 5-10% agarose pads. Agarose included 0.002% 1-phenoxy-2-propanol (Janssen Chimica). Worms were cover slipped and the slide sealed in Vaseline to prevent evaporation. Axotomy was performed as described above, and time lapse images were collected (Lasersharp 2000) every 5 min. over a Z range of 10-15 μm at 0.1 μm/pixel resolution. Maximum projections of each time point were exported to ImageJ for analysis.

### Example 1

### Axon Regeneration Requires A Conserved MAP Kinase Pathway

Regeneration of injured neurons has the potential to restore function, but most neurons regenerate poorly or not at all. The failure to regenerate in some cases can be attributed to a lack of activation of cell-intrinsic regeneration pathways. Thus, the components of these pathways can be targeted for the development of therapies that can restore neuron function after injury or disease. Here, DLK-1, MKK-4, and p38/PMK-3 were shown to comprise a conserved MAP kinase pathway that facilitates regeneration in C. elegans motor neurons. Loss of this pathway completely eliminates regeneration, while activating these genes improves regeneration. Further, these proteins regulate both initiation of regeneration and the later step of growth cone migration. Despite its critical functions in regeneration, the DLK-1 pathway is not required for axon outgrowth and wiring during development. It is concluded that activation of this MAP kinase cascade by axon injury is required to switch the mature neuron from an aplastic state to a state capable of robust growth. Thus, the complex activation of p38 and Jnk MAPK pathways is essential for axon regeneration. Controlling activation through these pathways can force “old” non-regenerating neurons into neurons with embryonic-like regeneration capabilities. Knowledge of the complex MAPK pathways controlling axon regeneration in C. elegans can be used to activate “embryonic-like” regeneration abilities in old non-regenerating neurons.

Severed neurons can regenerate. After axons are cut, neurons can extend a new growth cone from the axon stump and attempt to regrow a normal process. Most invertebrate neurons can regenerate, as can neurons in the mammalian peripheral nervous system. By contrast, neurons in the mammalian central nervous system have limited regenerative capability (Case et al. 2005). To successfully regenerate, the neuron must recognize that it has been injured and activate regrowth. Regeneration is thought to be initiated by signals arising from the injury, including calcium spikes and the retrograde transport and nuclear import of regeneration factors (Rossi et al. 2007). These mechanisms lead to increased cAMP levels, local and somatic protein synthesis, and changes in gene transcription, that in turn promote remodeling of the cytoskeleton and plasma membrane at the site of injury. The ability of specific neurons to regenerate is determined in part by the balance between pro-regeneration signals and cellular pathways that inhibit regeneration. For example, regeneration in the mammalian CNS is inhibited by extrinsic signals from myelin and chondroitin sulfate proteoglycans (CSPGs); these signals activate pathways in the damaged neuron that prevent regrowth (Liu et al. 2006). But CNS regeneration can be achieved even in the presence of inhibitory signals. A conditioning lesion to a peripheral process results in increased regeneration of the CNS branch of dorsal root ganglion neurons, presumably by triggering injury signals that result in a overall increase in regenerative potential (Neumann and Woolf, 1999). Thus, intrinsic regeneration signals can influence regenerative success, and these signaling processes represent potential targets for therapies to enhance regeneration.

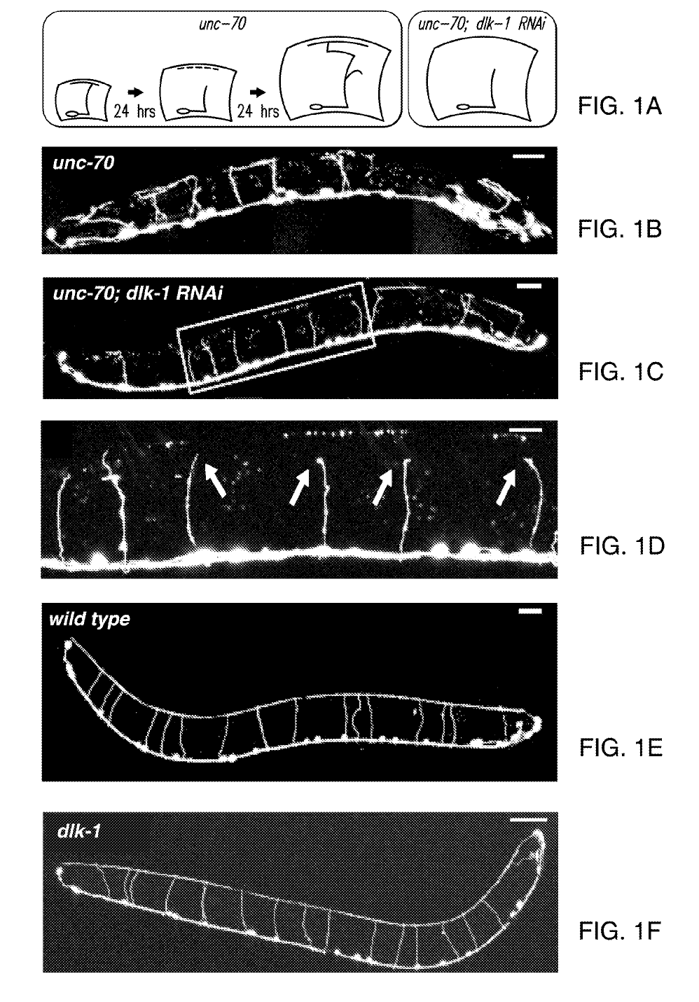

The MAP kinase kinase kinase (MAPKKK) dlk-1 as essential for regeneration in the course of a large screen for genes required for regeneration. This screen was conducted in a (3-spectrin mutant background. β-spectrin is encoded by the unc-70 gene in C. elegans (Hammarlund et al. 2000). Neurons in unc-70(s1502) mutant animals develop normally but are fragile. After these animals hatch, their axons break due to mechanical strain induced by normal locomotion. The GABA inhibitory motor neurons respond to breaks by initiating a growth cone and growing toward their post-synaptic targets in the dorsal cord (Hammerlund et al. 2007). Axon guidance during regeneration is imperfect, resulting in axons in mature animals with branching and other abnormalities (FIG. 1A, left and 1B). Moreover, new breaks are constantly occurring in the unc-70 control animals, such that in L4-stage an average of only 9 GABA axons extend to the dorsal cord, compared to 16 counted in the wild type (FIG. 1B). RNA interference of dlk-1 eliminates regeneration and only the broken stumps remain (FIG. 1A, right and IC). An average of only 4 axons reached the dorsal cord (FIG. 1C). The remaining GABA axons in the knockdown animals appeared healthy, but terminated in undifferentiated stumps (FIG. 1D). Neither unc-70 (6) nor dlk-1 are essential for axon outgrowth during development of the GABA neurons (Nakat et al. 2005) (FIGS. 1E and F). The unc-70 dlk-1 synthetic phenotype for axon morphology suggests that dlk-1 can function specifically in regeneration.

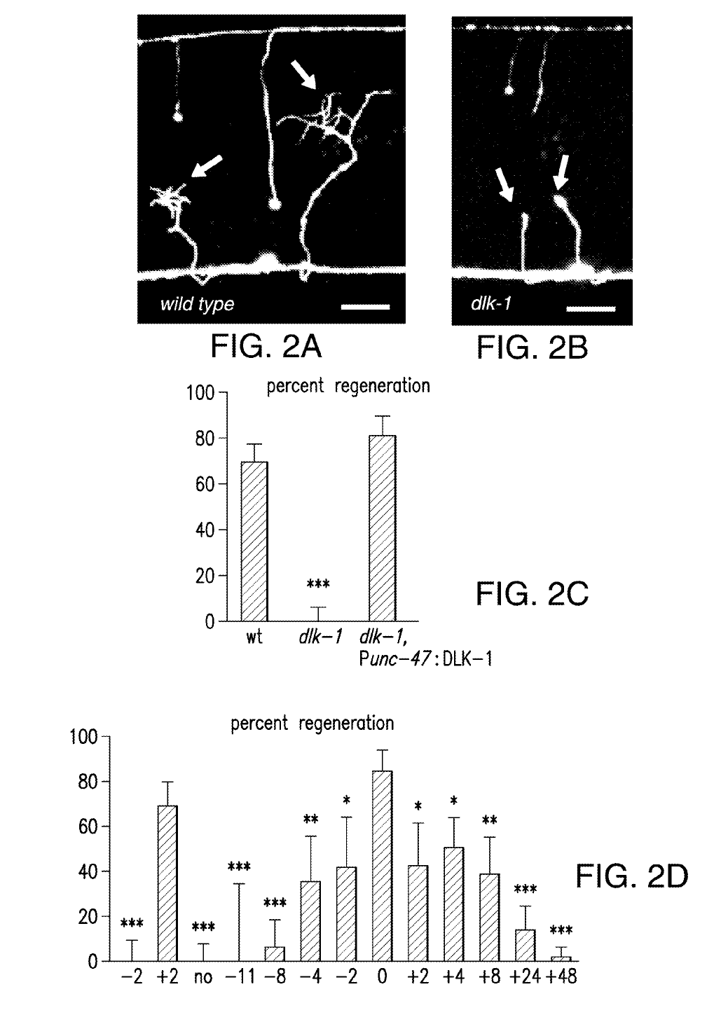

To demonstrate that dlk-1 plays a specific role in regeneration, independent of unc-70, laser axotomy was used to trigger regeneration. The GABA motor neurons can regenerate after laser axotomy (Yanik et al. 2004). A pulsed 440 nm laser was used to cut axons (Wu et al. 2007). In L4-stage wild-type animals, 70% of severed axons initiated growth cones within 24 hours after axotomy (73/105 axons; FIGS. 2A and C. See Table 1 for complete genotype and number of animals in each experiment). But when axons were cut in dlk-1 (ju476) null mutants, growth cones were never observed (P<0.0001; 0/69 axons; FIGS. 2B and C). These severed neurons appeared healthy after surgery: both the stump of the remaining axon and its cell body showed no decrease in GFP expression or other signs of injury. Nevertheless, these neurons failed to regenerate. To test whether regeneration was merely delayed, some severed axons for 5 days was monitored: regeneration still was not observed. Thus, dlk-1 is essential for axon regeneration after spontaneous breaks and after laser surgery, but is dispensable for axon outgrowth during development.

The L4-stage GABA motor neurons are of two types, DD and VD, with similar morphologies but opposite polarities (White et al. 1986). Both types of neurons have their cell bodies on the ventral side and extend processes in the dorsal and ventral cords. The DD neurons are presynaptic on the dorsal side, while the VD neurons are postsynaptic on the dorsal side. Axotomy at the midline therefore severs the presynaptic region of the DD neurons and the postsynaptic region of the VD neurons. Despite this difference in polarity, regeneration was similar in DD and VD neurons (Wu et al. 2007). Further, it was found that dlk-1 is required for regeneration of both neuron types. Thus, dlk-1 is required for regeneration of both presynaptic and postsynaptic processes.

Mosaic experiments demonstrate that the DLK-1 protein acts in the damaged cell rather than in the surrounding tissue. To determine whether DLK-1 acts cell-autonomously to promote regeneration, DLK-1 was expressed under the GABA-specific promoter Punc-47 in the dlk-1 null background. Neurons were severed by laser surgery and regeneration assayed after 18-24 hours. It was found that expressing DLK-1 in the GABA neurons restored regeneration to dlk-1 null mutants (P=0.13; 43/53 axons; FIG. 2C).

To mediate regeneration, DLK-1 is required at the time of injury rather than during development of the nervous system. The DLK-1 protein was expressed at different times using the heat shock promoter Phsp-16.2. dlk-1 mutant animals carrying the Phsp-16.2:DLK-1 construct showed no regeneration without heat shock, similar to dlk-1 mutants alone (P<0.0001; 0/55 axons; FIG. 2D ‘no’). By contrast, heat shock applied immediately following surgery resulted in robust regeneration, similar to wild-type animals (P=0.21; 21/25 axons; FIG. 2C ‘0’). Importantly, applying heat shock two hours before surgery to dlk-1 animals that did not have the Phsp-16.2:DLK-1 construct did not improve regeneration, demonstrating that the improvement was due to the specific expression of DLK-1 rather than a general response to heat shock (P<0.0001; 0/46 axons). Heat shock also did not affect the ability of wild-type animals to regenerate (P=1.0; 35/51 axons). These experiments demonstrate that DLK-1 expression at the time of injury is sufficient for regeneration. To further characterize the temporal requirements for DLK-1, the heat shock was varied relative to the time of surgery (L4 stage). Applying heat shock hours before or hours after surgery resulted in less regeneration compared to heat shock at the time of surgery, and when heat shock was applied either 11 hours before or 48 hours after surgery, little or no regeneration was observed (−11 hours: P<0.0001; 0/9 axons. +48 hours: P<0.0001; 1/96 axons). Thus, DLK-1 must function within a short temporal window near the time of surgery to mediate regeneration, rather than establishing a permissive state for regeneration during development. These data show that DLK-1 signaling must coincide with other early pro-regeneration signals, such as calpain activation (Gitler et al. 2002) or cAMP elevation (Chierzi et al. 2005; Neumann et al. 2002; Qui et al. 2002), for regeneration to occur.

DLK-1 is required for the formation of the growth cone rather than the early step of filopodial extension. Time-lapse microscopy to monitor morphological changes was used in wild-type and dlk-1 mutant axons after surgery. It was found that in wild-type animals, newly-severed axons repeatedly extend short, transient filopodia from the axon stump (FIGS. 3A and G). The first filopodium appears with an average delay of more than three hours (198.2±26.8 minutes) after surgery, and the earliest appearance was at 2 hours after surgery (115 minutes; FIGS. 3G and H). The stump continues to initiate filopodia (average rate=0.56±0.08 filopodia/hour). In animals that successfully initiate regeneration, a single filopodium eventually persists and is transformed into a growth cone. Growth cone formation in wild-type animals occurs after an average delay of 7 hours (426.2±36.6 minutes) after surgery (FIG. 3J). In dlk-1 mutants, transient filopodia appear at approximately the same time and the same rate as in the wild type (first filopodium: P=0.29; 155.6±28.7 minutes; rate: P=0.54; 0.49±0.08 filopodia/hour; FIGS. 3B and G-I). However, growth cones were never observed in these mutants. These data demonstrate that DLK-1 is required at an early step of regeneration, during the transformation of an exploratory filopodium into a growth cone.

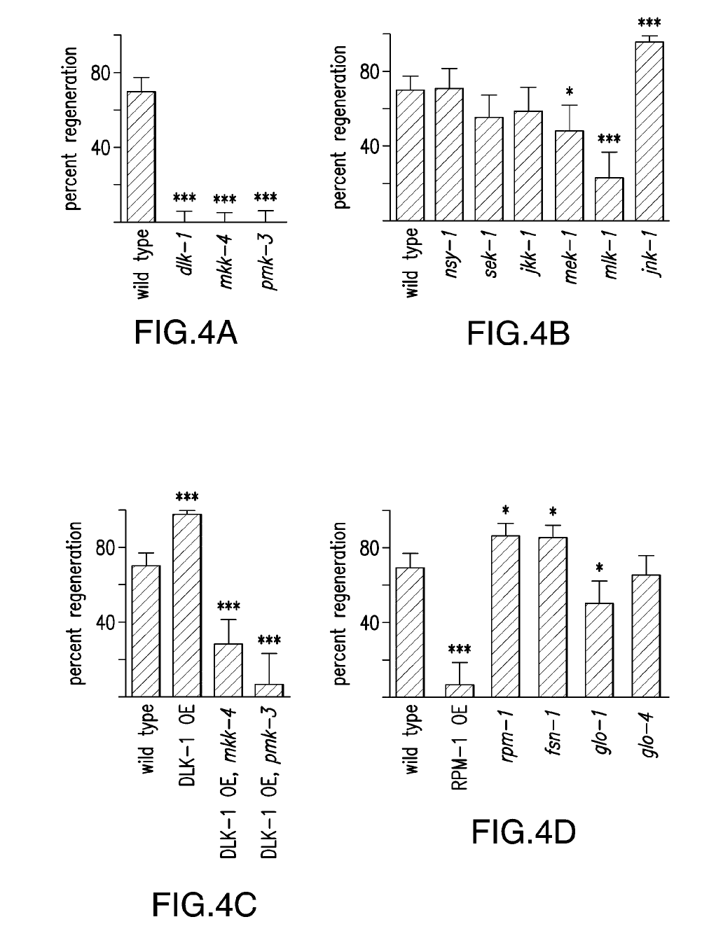

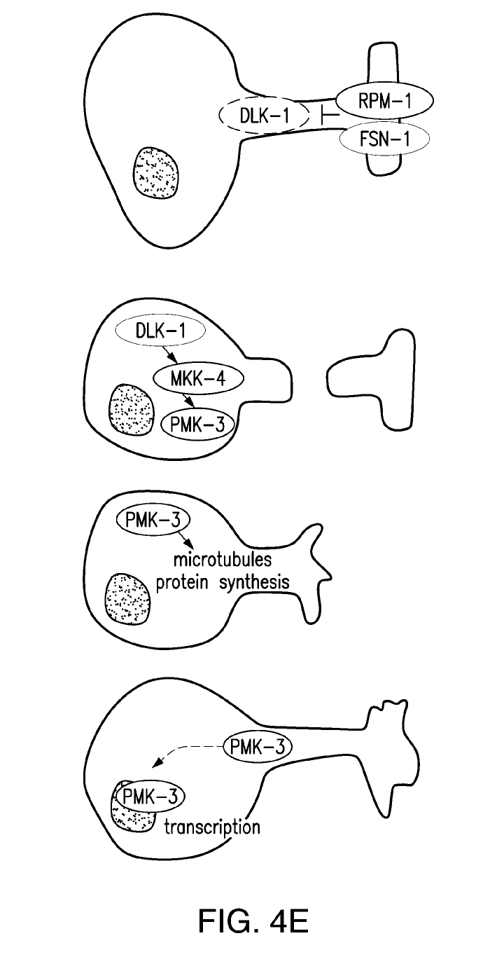

Increased expression of DLK-1 in wild-type animals accelerates the formation of growth cones and improves migration success. DLK-1 was over-expressed in GABA neurons using the Punc-47 promoter and monitored regeneration using time-lapse microscopy. The time of growth cone initiation by axon stumps was advanced relative to the wild type (P<0.0001; average delay=91.25±16.33 minutes; FIG. 3C, D). Also, more axons initiated growth cones (P<0.0001; 42/43 axons; FIG. 4C). In addition to these effects on growth cone initiation, DLK-1 over expression improved growth cone performance. In wild-type animals, regenerating growth cones often have a branched, dystrophic morphology. Dystrophic growth cones migrate poorly, and most never reach the dorsal nerve cord in 24 hours (3/73; FIGS. 3E and K). These dystrophic growth cones bear a striking resemblance to dystrophic growth cones observed in failed regeneration in the mammalian CNS (Silver et al. 2004). By contrast, regenerating growth cones in neurons that over express DLK-1 have a compact shape, similar to growth cones observed during initial axon development (Knobel et al. 1999). These compact growth cones were much more likely to reach the dorsal nerve cord (P<0.0001; 21/42; FIGS. 3F and 3K). Thus, DLK-1 acts at two steps of regeneration: it is required for growth cone formation, and it also controls growth cone morphology and behavior.

DLK-1 functions in a MAP kinase signaling cascade that also includes the MAP kinase kinase (MAPKK) MKK-4, and the p38 MAP kinase (MAPK) PMK-3 (Nakata et al. 2005). It was tested whether this entire MAP kinase signaling module functions in regeneration by examining null mutants in mkk-4 and pmk-3. Like dlk-1, neither of these mutants has appreciable defects in axon outgrowth during development. But after axotomy, both mutant strains completely fail to initiate regeneration (mkk-4: P<0.0001; 0/76 axons; pmk-3: P<0.0001; 0/69 axons; FIG. 4A). These data show that MKK-4 and PMK-3 are the downstream targets of DLK-1 for regeneration. Inhibition of p38 also reduces regeneration of cultured vertebrate neurons (Verma et al. 2005), suggesting that the function of p38 MAP kinases in regeneration is conserved.

A sampling of C. elegans MAP kinase components were tested and it was found that mutations in these genes did not eliminate regeneration (FIG. 4B). Initiation of regeneration was not affected by loss of the MAPKKK nsy-1 or its target MAPKK sek-1 (nsy-1: P=1.0; 34/48 axons; sek-1: P=0.08; 28/51 axons). Loss of the MAPKK jkk-1 also did not affect regeneration (P=0.26; 27/46 axons).

However, results with mlk-1, mek-1, and jnk-1, and the residual axon regeneration seen in mkk-4 and pmk-3 when over expressing DLK-1 suggested that other MAPK pathways can play a role in axon regeneration. Both PMK-3 (p38a) and KGB-1(jnk-like) are essential for successful axon regeneration. The MAKP phosphatase vhp-1 and the JSAP unc-16 have roles in the regulation of these pathways. Furthermore, loss of the MAPKKK mlk-1 significantly reduced initiation of regeneration (although some regeneration still occurred), as did loss of its downstream target mek-1 (mlk-1: P<0.0001; 12/52 axons; mek-1: P=0.017; 22/46 axons). For example, JNK signaling is critically in C. elegans. Surprisingly, loss of the MAPK jnk-1 increased initiation of regeneration (P<0.0001; 65/68 axons). Axon regeneration is improved in jnk-1, and is completely blocked (100%) in neurons over expressing JNK-1. Axon regeneration is also completely blocked (100%) in kgb-1 (jnk-like), while it is dramatically improved (near 90%) in kgb-2 (jnk-like). Both PMK-3 (p38a) and KGB-lank-like) are essential for successful axon regeneration.

Thus, while the DLK-1/MKK-4/PMK-3 MAP kinase cascade is absolutely required to initiate regeneration, other MAP kinase pathways also regulate this'process. Consistent with these data, mutations in mkk-4 or pmk-3 did not completely eliminate the stimulation of regeneration by DLK-1 over expression, suggesting that crosstalk between MAP kinase modules can contribute weakly to regeneration (DLK-10E; mkk-4: P<0.0001; 14/50 axons; DLK-10E; pmk-3: P<0.0001; 3/46 axons; FIG. 4C). However, the modest phenotype of other MAP kinase mutants, and the inability of DLK-1 over expression to bypass the requirement for mkk-4 and pmk-3, show that the DLK-1/MKK-4/PMK-3 module is the major MAP kinase pathway for axon regeneration.

DLK-1 is a MAPKKK that has been previously characterized for its involvement in synapse regulation together with RPM-1/Highwire. DLK-1-family MAPKKKs are activated by homodimerization and autophosphorylation (Gallo et al. 2002). In C. elegans, DLK-1 activity is negatively regulated by targeted destruction via RPM-1, an E3 ubiquitin ligase PHR protein (Nakata et al. 2005). RPM-1 is a large protein with a RING finger domain (Schaefer et al. 2000; Zhen et al. 2000) that stimulates the ubiquitination and degradation of DLK-1 (Nakata et al. 2005). It was found that over expression of RPM-1 reduced regeneration after surgery to levels similar to dlk-1, mkk-4 and pmk-3 loss-of-function mutants (P<0.0001; 3/43 axons; FIG. 4D). Conversely, initiation of regeneration was enhanced in rpm-1 mutant animals, similar to the increase in initiation caused by DLK-1 over expression (P=0.030; 44/51 axons). Initiation of regeneration was also increased in animals lacking FSN-1, an F-box protein that functions with RPM-1 to promote DLK-1 degradation (Grill et al. 2007) (P=0.016; 56/65 axons). However, loss of GLO-1/Rab or GLO-4/Rab GEF, which mediate ubiquitin-independent functions of RPM-1 (Grill et al. 2007), did not have strong effects on regeneration (glo-1: P=0.016; 33/65 axons; glo-4: P=0.73; 39/59 axons). These results show that RPM-1-mediated degradation is a critical regulator of DLK-1 function in regeneration.

What stimulates DLK-1 function when an axon breaks? One possibility is that constitutive degradation of DLK-1 by RPM-1 is disrupted and DLK-1 protein levels rise. In the simplest model, the axon break physically interrupts trafficking of DLK-1 to sites of degradation by RPM-1 in the distal regions of the axons. DLK-1 accumulation in the injured neuron then leads to homodimerization and activation, followed by activation of the downstream targets MKK-4 and PMK-3 (FIG. 4E). Since RPM-1 is enriched at pre-synaptic terminals (Zhen et al. 2000), this model provides a simple way for axotomy to activate DLK-1. But non-synaptic signals must also activate DLK-1 after axotomy, since severing ‘dendritic’ regions from the VD cell body also stimulates regeneration. Moreover, the vertebrate homolog of RPM-1, Phrl, is not solely localized at synapses but is found throughout axon shafts (Lewcock et al. 2007). RPM-1 inactivation by damage might be ubiquitous. Alternatively, RPM-1 is not the sensor of axon injury, but rather other regulatory mechanisms activate DLK-1, such as scaffolding proteins like Jip1 (Nihalani et al. 2001), phosphatases, such as PP1, PP2a, and calcineurin (Mata et al. 1996) or regulators of the proteasome (Daviau et al. 2006).

Why is the DLK-1 pathway essential for regeneration (FIG. 2)? DLK-1 is not required for axon development (FIG. 1F). During development, multiple factors maintain neurons in a state of active growth (Goldberg et al. 2003). Once axons have reached their target and begun synaptogenesis, termination of these signals by mechanisms like RPM-1 can down regulate growth to allow synapse maturation and to stabilize neuronal architecture. Indeed, mutations in RPM-1 or its homologs cause overgrowth of axons in worms (Schaefer et al. 2000), aberrant sprouting in Drosophila (Collins et al. 2006), and aberrant growth cone initiation on axon shafts in mouse (Lewcock et al. 2007). The strict requirement for the DLK-1 pathway in regeneration shows that mature neurons have intrinsic barriers to growth that are not present during development. Thus, active signaling via DLK-1, MKK-4, and PMK-3 is required to drive the neuron back to its prelapsarian, embryonic state.

How does the MAP kinase PMK-3 stimulate regeneration? The first sign of regeneration occurs about 3 hours after the axon is broken, when the stump extends filopodia. The DLK-1 pathway is not required for this early step of regeneration (FIG. 3), but rather is first required for growth cone formation about 7 hours after a break occurs—a process likely to be mediated by the polymerization of microtubules. Activated p38 MAP kinase regulates microtubule dynamics (Lewcock et al. 2007), and microtubule remodeling is required for growth cone initiation during regeneration (Erez et al. 2007). Further, defects in microtubule dynamics contribute to the axon outgrowth phenotype of Phrl mutant mice (Lewcock et al. 2007). Activated p38 can also control other targets that facilitate axon regeneration. p38 regulates local protein synthesis (Campbell et al. 2003), which is required for regeneration (Verma et al. 2005). p38 is also likely to have functions in the nucleus, since it contributes to injury-induced changes in gene transcription (Zrouri et al. 2004). Activated p38 can reach the nucleus by retrograde transport: retrograde transport in general is critical for regeneration (Hanz et al. 2006) and transport of activated MAP kinases from axons to the cell body following axotomy has been observed in Aplysia sensory neurons (Sung et al. 2001) and in rodent sciatic nerves (Perlson et al. 2005; Reynolds et al. 2001). Thus, regeneration can require activated PMK-3/p38 at the site of the break to regulate microtubule stability and protein expression, and also require PMK-3 to traffic to the nucleus to regulate gene transcription (FIG. 4E). The DLK-1 signaling pathway thus provides a critical link between axon injury and the process of regeneration.

The present studies demonstrate that there is a profound age dependent decline in axon regeneration in C. elegans neurons. Several aging mutants were tested to determine the effects of JNK-1 and DLK-1 on axon regeneration. daf-2 and daf-16 (FOXO) are key members of the IGFR signaling pathway that affect aging. In daf-2, which has an increased lifespan, axon regeneration is proportionately increased, while axon regeneration in daf-16 is reduced. There does not appear to be a perfect correlation between aging and axon regeneration. For example, while the over expression of JNK-1 is reported to increase lifespan, the present studies show that such overexpression completely blocks axon regeneration. Both the JNK and p38 pathways also directly influence the AKT/FoxO and mTor pathways that are involved in aging, and that have been shown can strongly influence axon regeneration.

### Example 2

### Neuronal Regeneration Screening, and the p38 MAPK Pathway

Neuronal regeneration has been studied in humans and other vertebrate model systems for over 100 years and yet there is still no comprehensive molecular model, nor an effective treatment for axotomy due to injury or disease (Horner et al. 2000; Case et al. 2005; Goldberg 2004; Rossingnol et al. 2007; Silver et al. 2004). In vivo and in vitro model systems have been developed that have focused on identifying the molecular differences between vertebrate neurons that can and cannot regenerate, e.g., PNS versus CNS and embryonic versus adult, or between animals that exhibit major differences in their regenerative capacities, e.g., salamander versus mouse. Many exciting discoveries have been made that illustrate the importance of both the extracellular molecular environment and the intrinsic cellular “state” of the neuron. The classic experiments by Aguaya's group in the 1980s showing that adult CNS neurons can indeed regenerate if given the right environment, and the identification of many inhibitory molecules associated with CNS myelin and the glial scar stand out as turning points reigniting interest in potential therapies for axotomy (Rossingnol et al. 2007). Genetic and cellular studies identifying genes regulating neuron development, motility, and pathfinding have added to the excitement and provided many new environmental and cell intrinsic molecular signaling pathways as potential regulators of neural regeneration (Case et al. 2005).

Surprisingly, the powerful genetic model systems used so successfully to study body pattern formation, programmed cell death, neural development and many other biological problems have not been used to study neuronal regeneration. It has been difficult to devise a robust screening assay for neural regeneration in either D. melanogaster (Leyssen et al. 2007) or C. elegans (Yanik et al. 2004; Wu, Z. et al. 2007; Gabel et al. 2008). Recently, an observation was made that now makes it possible to screen for genes that effect regeneration in C. elegans (Hammarlund et al. 2007). It was discovered that embryonic neurons lacking β-spectrin develop normally, but after hatching undergo a movement-induced axotomy followed by regeneration. This is a very dramatic and robust phenotype, with most commissural axons in each animal breaking and regenerating before the animal even reaches adulthood. There is a progressive failure of regeneration as each cycle of axotomy and regeneration takes place so that the adult displays a severely abnormal nervous system (Hammarlund et al. 2000). This well characterized regeneration phenotype in C. elegans mimics the phenotype of mammalian neurons in response to axotomy (Hammarlund et al. 2007; Luo et al. 2005). RNAi knockdown can be used to unbiasedly assay the effect of every gene in the worm genome on the process of neuronal regeneration.

Until recently, RNAi knockdown by feeding only worked for a small percentage of genes expressed in post embryonic neurons (Kamath et al. 2003). Neurons in C. elegans seemed to be very resistant to RNAi for unknown reasons. Since then, genetic mutations have been identified that sensitize neurons to RNAi (Kennedy et al. 2004; Wang et al. 2005; Lehner et al. 2006). One of these sensitized genetic backgrounds has been used and validated in a successful large scale RNAi screen for genes that effect synaptic transmission (Sieburth et al. 2005). It has been demonstrated that this technique can be used to screen for mutations that affect regeneration of neurons lacking β-spectrin.

Spectrin is an essential component of the membrane cytoskeleton in metazoans and its functional role mediating mechanical support has been well characterized in the red blood cell (Bennett et al. 2001). It is abundantly expressed in the nervous system, both during development and in the adult, but its role in neurons is not well understood. There is evidence that spectrin can play a role in growth cone motility, axon and dendrite stability, and synaptic function (Bennett et al. 2001). In C. elegans, the unc-70 gene encodes the single (1-spectrin subunit. The initial characterization of the unc-70 mutant described a very uncoordinated animal with a severely abnormal nervous system (Hammarlund et al. 2007). The nervous system defects included neurons with premature axon termination, excess axon branching, and the presence of growth cones in the adult. These defects are consistent with several different proposed functions of β-spectrin, including a role in growth cone motility and membrane stability. The striking appearance of growth cones in the adult caused a first look for growth cone motility defects as the primary cause of the nervous system abnormalities in the β-spectrin mutants.

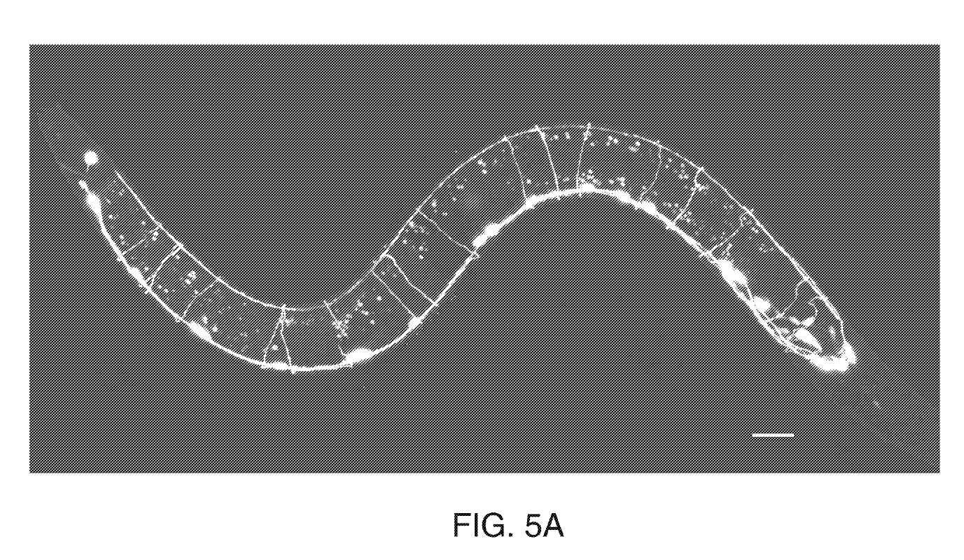

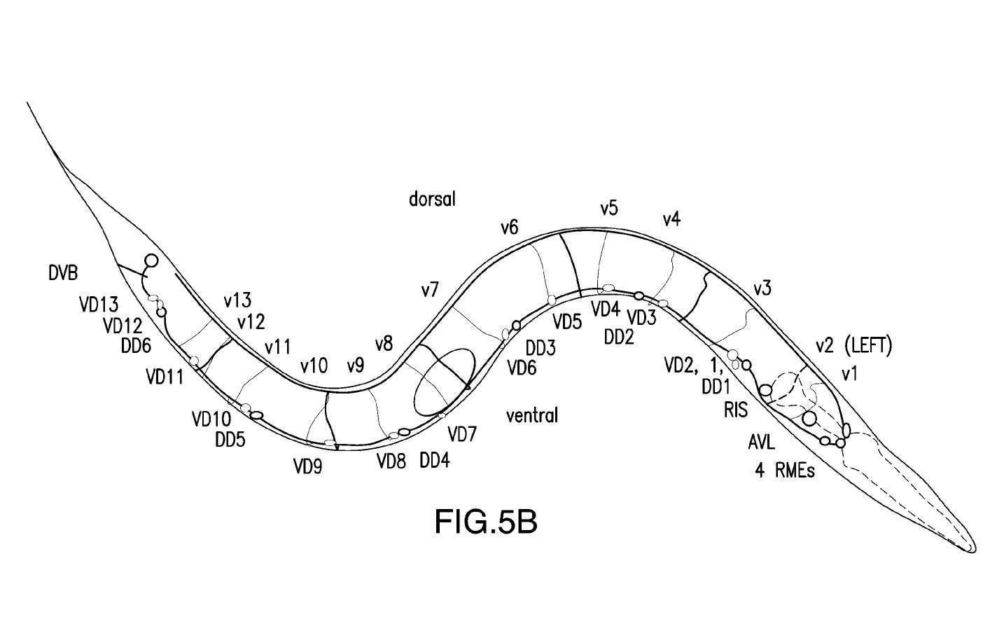

In previous studies, the growth cones of the post embryonic GABAergic VD neurons using the p-unc-47::GFP N-terminal transcriptional fusion as an in vivo marker were imaged (Knobel et al. 1999). This marker is expressed in the cytoplasm of all GABAergic neurons soon after their birth (FIG. 5) (Schuske et al. 2004). The 6 DD neurons are born during embryogenesis, while the 13 VD neurons are born post embryonically. Both DD and VD neurons extend growth cones first anteriorly along the ventral nerve cord and then dorsally as commissural axons to the dorsal nerve cord where they extend anterior and posterior branches. The VDs extend their growth cones during the late L1 stage where they can be easily imaged. The stereotyped behavior of wild type VD growth cones has been characterized as they migrate along their ventral to dorsal pathway (Knobel et al. 1999). VD growth cones were imaged in (3-spectrin mutants and were surprised to find their behavior and morphology completely normal (Hammarlund et al. 2007). The rate of growth and the characteristic shape changes seen in the growth cones of the unc-70 animals were indistinguishable from the wild type behaviors of growth cones. More importantly, abnormal behaviors that correlated with the severely abnormal nervous system of the adult unc-70 animals were not observed.

The severely abnormal unc-70 nervous system must arise sometime after initial neuron development, but before adulthood. To determine exactly when and how the nervous system defects arise, neurons were studied starting at the time of their embryonic development and following them through sequentially older animals. The DD GABAergic neurons were chosen rather than the VDs because the DDs arise embryonically and can be followed as individually identifiable neurons for a longer period of time. Embryonic DD neurons grow out and form normal commissural and dorsal nerve cord projection in unc-70 animals when compared to wild type animals. Just as with the VDs, the pattern of embryonic DD growth cone morphologies and axons was virtually indistinguishable from the wild type (Hammarlund et al. 2007).

DD neurons were next studied at hatch (7-8 hours after the DDs extend embryonically) and then again at 24 hours post hatch. Here the results were remarkably different. DD neurons at hatch had 26% more defects than embryonic DD neurons, while at 24 hours post hatch that number increased to 60%. The defects observed included aberrant branching, growth cones, and “broken” axons. These types of defects were never seen in wild type embryos. These data demonstrate that DD neurons develop normal axon projections embryonically and then display progressively more axon defects with age. However, because different animals (DD neurons) were imaged at each different time point (embryo, hatch, 24 hours post hatch), how the axonal defects arose was not be explained how the axonal defects arose, and if there was any relationship among the different defects that were observed. To understand how the axonal defects in the DD neurons arise as the animal ages were looked at in a “longitudinal” study. In these experiments DD neurons were imaged at hatch and then every 24 hours for up to 4 days in individual animals. The morphological changes that occurred in a single identified unc-70 DD neuron as it aged were then determined.

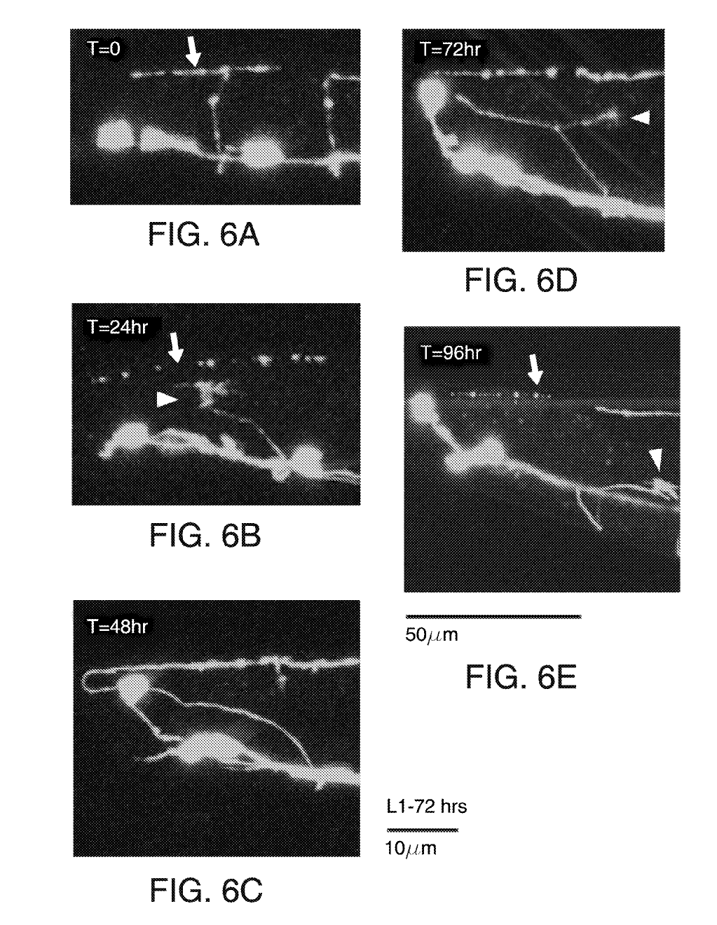

FIG. 6 shows a single DD neuron that has been imaged at hatch, 24, 48, 72, and 96 hours. The DD neuron has a normal morphology at hatch, but by 24 hours the commissural process has broken and re-initiated a growth cone (arrowhead), while the distal process is degenerating (arrow). At 48 hours that DD has regenerated a process back to the dorsal nerve cord, but by an aberrant pathway. It has extended much farther posterior than normally seen for a DD neuron and extends a single anterior process in the dorsal nerve cord rather than a T shaped process. At 72 hours, the DD commissural axon has broken again and it appears as if aberrant regeneration occurred to give a branched commissure. The dorsal nerve cord process again seems to be degenerating. At the last time point taken at 96 hours, the dorsal nerve cord process has completely degenerated and mostly been cleared (arrow), the left hand commissure branch has retracted or degenerated and the right hand branch again re-initiated a growth cone extending dorsal and anterior (arrowhead).

The observations of single DD neurons in these longitudinal studies now allow for the description of the origin of the severe nervous system phenotype in the β-spectrin mutants. Neurons are born and extend growth cones to their targets. There is no defect in either growth cone motility, pathfinding, nor target recognition. Subsequent to the development of normal morphology, the axon breaks, the distal process degenerates by a process that closely resembles Wallerian degeneration (Hammarlund et al. 2007, Luo et al. 2005), and the proximal process regenerates by extending a new growth cone towards its target. Regeneration is often successful, but is clearly more error prone than embryonic development, thus leading to the appearance of aberrant branches. This cycle of outgrowth, axotomy, and regeneration with accumulated errors leads to the severe nervous system phenotype in the adult animals of the unc-70 genotype. Therefore, the primary defect in β-spectrin mutants is axotomy.

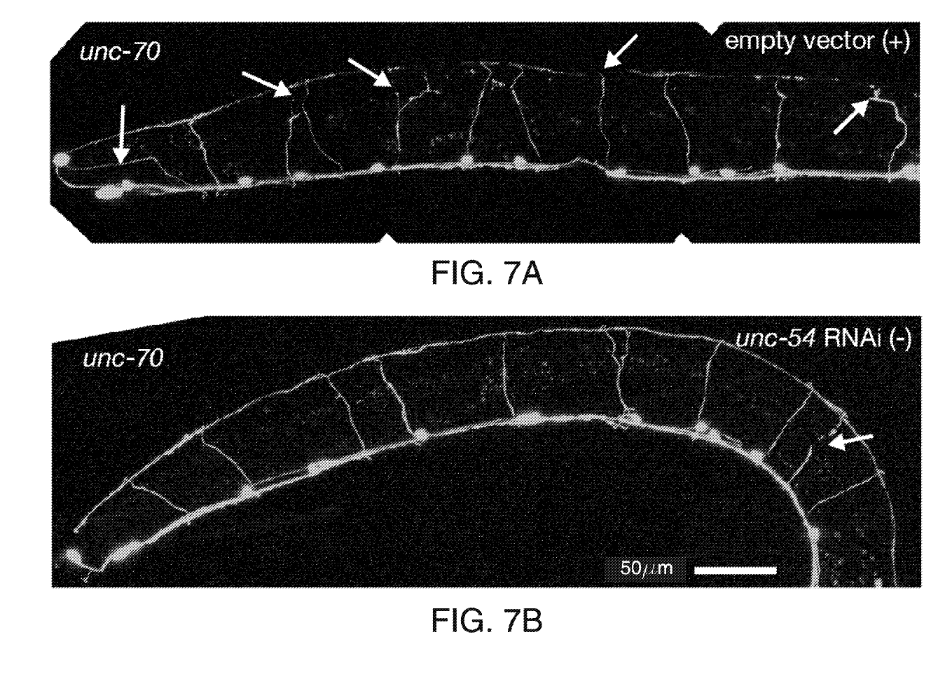

Why do axons of neurons missing β-spectrin break? One explanation is that β-spectrin can be important for the membrane addition associated with interstitial axon growth during animal growth. Another explanation is that β-spectrin can provide the membrane with the cytoskeletal strength and elasticity needed to resist mechanical stress associated with animal movement. This latter explanation is consistent with β-spectrin's classic role in the red blood cell (Bennett et al. 2001). Neurons are unique in having extended axonal and dendritic processes that can be particularly susceptible to defects in both rapid membrane addition during animal growth and mechanical stress associated with animal movement. These two explanations by inhibiting a muscle myosin gene (unc-54) by the technique of RNAi (Simmer et al. 2005). Inhibiting muscle myosin by RNAi completely paralyzes worms and in addition leads to larger animals. If muscle contraction and mechanical strain cause axotomy, then suppression of the phenotype in animals that grow up paralyzed is expected. If animal growth causes axotomy, then in animals that grow larger the enhanced phenotype is expected. The result strongly supports the idea that muscle contraction and mechanical strain cause axotomy of neurons lacking β-spectrin (FIG. 7).

Paralyzing worms almost completely rescues the nervous system defects seen in unc-70 mutant animals (FIG. 7A vs. B). The controls show that neither RNAi of unc-54, nor the empty vector by itself affect the nervous system, however, unc-54 RNAi treatment of unc-70 animals dramatically decreases the number of axon defects compared to the unc-70 control animals (1.3 vs. 4.8 axon defects per animal, p<0.0001 two-tailed t test). To demonstrate that suppression of axon breakage was due to paralysis rather than a specific effect of unc-54 perturbation or the process of RNAi, the effect of the unc-22 mutation was tested (Hammarlund et al. 2007). unc-22 encodes the muscle protein twitchin, and mutant animals are unable to initiate coordinated movement or deep body bends. It was found that genetic loss of UNC-22 in the β-spectrin mutant background resulted in paralyzed animals with fewer neuronal defects than β-spectrin mutants alone (normal commissures: unc-70=2.7±0.4; unc-70; unc-22=6.7±0.5, N=10 each; p<0.0001, two-tailed t test). Thus, neurons in animals that lack β-spectrin are sensitive to strain caused by movement, rather than growth. These data show that β-spectrin does not function in the process of neuronal membrane addition during organismal growth. Rather, β-spectrin protects neurons against axotomy due to movement-induced strain.

In longitudinal studies, the detached fragments resulting from axotomy degenerated by disintegrating into multiple small particles, which were then cleared; this process resembles Wallerian degeneration (FIG. 6). To determine whether degeneration of severed axons in C. elegans proceeds by a Wallerian mechanism, the sensitivity of degeneration to two treatments that disrupt Wallerian degeneration was tested: over expression of nicotinamide mononucleotide adenylyltransferase (Nmnat) and loss of CED-1. Over expression of Nmnat1 in cultured mouse neurons inhibits the degeneration of detached axon fragments so that fragment persists longer. The two C. elegans homologs of Nmnat1 were over expressed in neurons. Similar to the results in mouse cells, over expression of Nmnat1 homologs (Nmnat OE) in β-spectrin mutants increased the survival of detached fragments and particles. CED-1 is required in C. elegans for the normal engulfment of cell corpses in programmed cell death, and is also required in Drosophila for eliminating axon fragments during Wallerian degeneration. It was found that eliminating CED-1 function in β-spectrin mutants resulted in an increase in detached fragments and particles. The absence of CED-1 resulted in a large increase in particles, showing that CED-1 can be required for engulfment of particles after the severed axon has been fragmented. The sensitivity of the degeneration to Nmnat and CED-1 shows that the removal of broken axons in β-spectrin mutants proceeds by a Wallerian mechanism, similar to that observed in mammalian neurons.

Together, these data show that β-spectrin is not essential for growth cone function or for axon elongation. Neurons grow normally in C. elegans animals that lack β-spectrin, both during the initial patterning of embryogenesis and later, in response to increases in the size of the worm. However, β-spectrin is essential for neurons to withstand acute tension generated by muscle contraction. In Drosophila neurons, β-spectrin functions in synapse stability and protein localization at the pre-synaptic terminal. In vertebrate neurons, spectrin functions in protein localization at the nodes of Ranvier. This single protein has multiple functions in neurons (Knobel et al. 1999). β-spectrin is one of the most abundant membrane proteins in the vertebrate brain, and is found in both axonal and dendritic processes of all neurons. Thus, elasticity of vertebrate neurons can also be determined in large part by β-spectrin. In sick or injured neurons, activated calpain targets the spectrin-based membrane skeleton for proteolysis and the data show that neurons depleted for spectrin are susceptible to axotomy. It is possible that axotomy due to reduction of spectrin function accounts for neurodegeneration in some human neurological diseases. Axon pruning in normal development can also occur by a degenerative mechanism, showing a function for targeted removal of the spectrin cytoskeleton (Luo et al. 2005).

In summary, β-spectrin is one of the most abundant membrane proteins in the mammalian nervous system and is found in all neurons associated with the membrane cytoskeleton of the cell body, axon and dendrites (Bennett et al. 2001). It has been shown that β-spectrin protects axons against strain-induced axotomy. Further, it has been shown that β-spectrin is not required for normal growth cone motility, pathfinding, or target recognition. These results by themselves can be important for understanding the origin and pathology of some human neurological diseases (Ikeda et al. 2006, Bauer et al. 2006). These results also allow for the assay for neuron regeneration in a model genetic organism. It has been shown that a high proportion of commissural axons undergo repeated cycles of axotomy, degeneration, and regeneration in the C. elegans unc-70 mutant. Gene knockdowns that specifically inhibit regeneration result in animals with quantitatively fewer or no intact commissures.

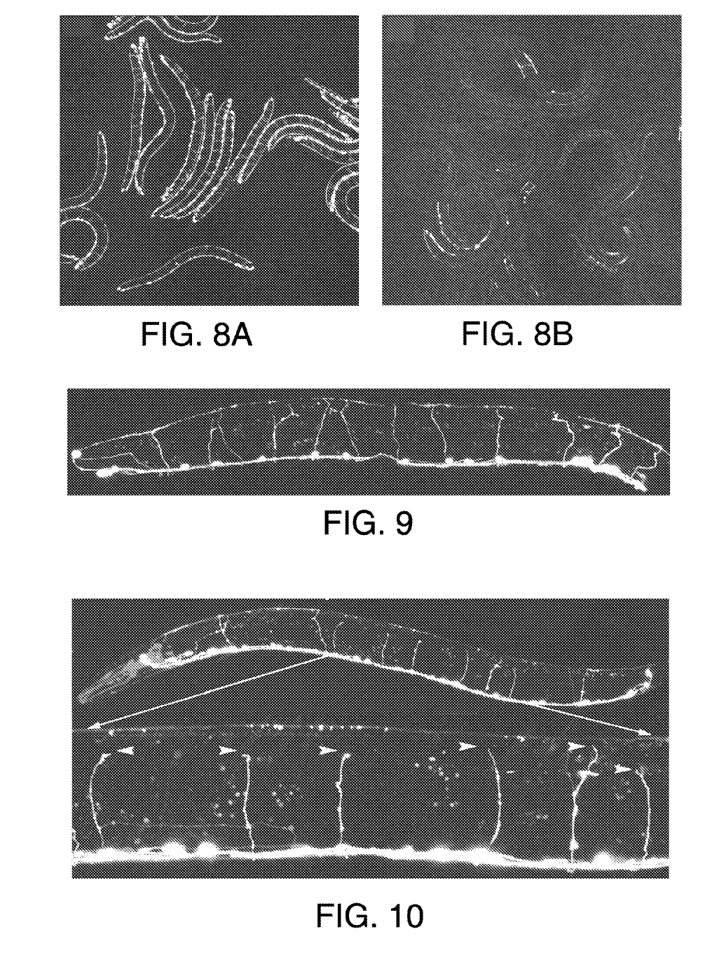

Several RNAi sensitized genetic backgrounds were tested to assay for effects on general health, GABA nervous system development, and effectiveness of GFP knockdown by bacterial feeding (Wang et al. 2005; Lehner et al. 2006; Sieburth et al. 2005). In FIG. 8 the level of GFP knockdown in the p-unc-47::GFP; eri-1; lin15b strain is shown. The strain was created for the regeneration screen is unc-70; p-unc-17::j3-spectrin; p-unc-17::mCherry; p-unc-47::GFP; eri-1; lin15b. This strain is healthy enough for use in the large-scale screen, and shows excellent and consistent GPF knockdown in GABA neurons. The phenotype of D motor neurons in unc-70 worms is variable and highly age dependent (Hammarlund et al. 2007). The D commissures in ten L4 worms from each RNAi plate were studied by observation under a compound scope at 400×. Worms are mounted on agar pads and cover slipped for high-resolution imaging. The number of D commissures that reach and extend in the dorsal nerve cord were studied. Any phenotypes associated with regeneration, i.e., growth cones, branching, and pathfinding errors were looked at. FIG. 9 shows an example of the nervous system phenotype of the unc-70; p-unc-17::β-spectrin; p-unc-17::mCherry; p-unc-47::GFP; eri-1; lin15b strain on the control RNAi plates. Branching defects, pathfinding errors, and gaps in the dorsal nerve cord are all characteristics of axons that have broken and regenerated. Over a 100 plates of control worms were screened, and it was consistently found that an average of 9-11 D commissures (averaged over ten L4 worms) exists.

The worm genome contains about 19,000 genes and most of these are represented by the RNAi clones of the Ahringer and Open Biosystems ORF-RNAi feeding libraries (Kamath et al. 2003; Rual et al. 2004; Johnson et al. 2005). 88 positives have been found that include transcription factors, cell surface or secreted molecules, cytoskeletal associated proteins, molecules involved in cellular metabolism, signaling proteins, and other uncharacterized proteins. (This is a hit rate of about 3-4% from the orthologs list and predicts about 100-200 genes can be identified affecting regeneration.) The screen is working reliably based. A small subset of genes that gave strong positives on both the initial screen and the secondary retest were then used, and the genetic nulls ordered. Axons were laser cut and neuron regeneration in “wild-type” neurons observed (Yanik et al. 2004; Wu, Z. et al. 2007; Gabel et al. 2008). This allowed for the testing of the regeneration phenotype of genetic nulls in an otherwise wild-type background, i.e., not in the unc-70; eri-1; lin-15b background. Many of the strong positives identified in the RNAi screen gave strong regeneration phenotypes when commissural axons were cut in the genetic nulls. Many of these show, as expected, some developmental phenotypes affecting growth cone guidance and motility.

FIG. 10 is an example of a strong positive (average of 5 or less D commissures) that has turned out to be quite interesting and illustrates the validity of the screen. The top panel A shows a L4 worm that has been growing on dlk-1 RNAi expressing bacteria. At first glance it looks pretty “normal” for an unc-70 worm (compare to the control). However, the closer look in the lower panel B shows exactly the characteristics predicted for genes that affect regeneration. The majority of D commissures end as axon “stubs” (arrowheads) and do not exhibit any growth cones or significant branching that show regeneration. Notice that there is only punctate GFP labeling in the dorsal nerve cord, while the mCherry labeling that represents the cholinergic neurons is continuous. This shows the RNAi knockdown of DLK-1 is specific for regeneration and not generally affecting neuron development or maintenance. DLK-1 is a MAPKKK that has been characterized for its involvement in synapse regulation together with RPM-1/Highwire (Nakata et al. 2005; Grill et al. 2007; Wu, C. et al. 2005). It was identified in C. elegans as a suppressor of rpm-1 (Nakata et al. 2005), but on its own has only subtle morphological and behavioral phenotypes. The morphology of the D motor neurons, based on p-unc-47::GFP expression, is wild type in the dlk-1 mutant. The D motor neurons were assayed for their ability to regenerate in the dlk-1 mutant to validate the results of the RNAi screen.

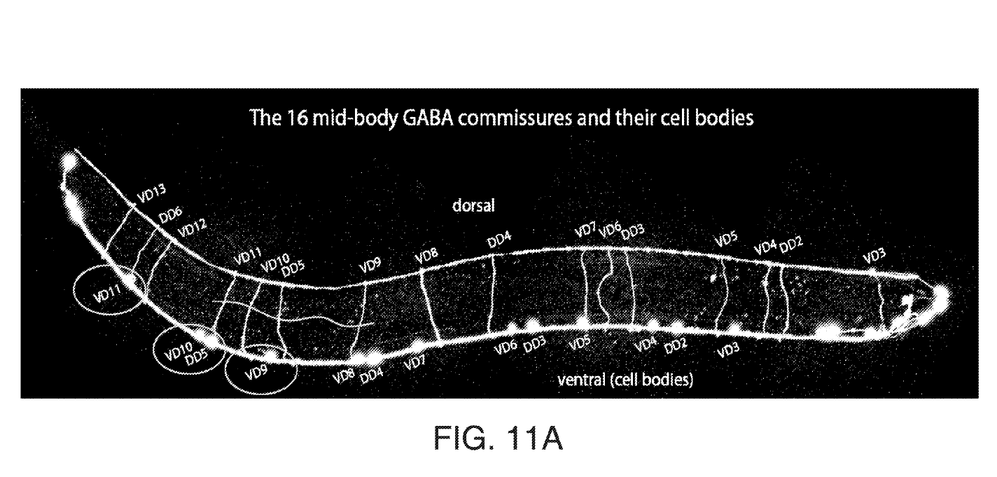

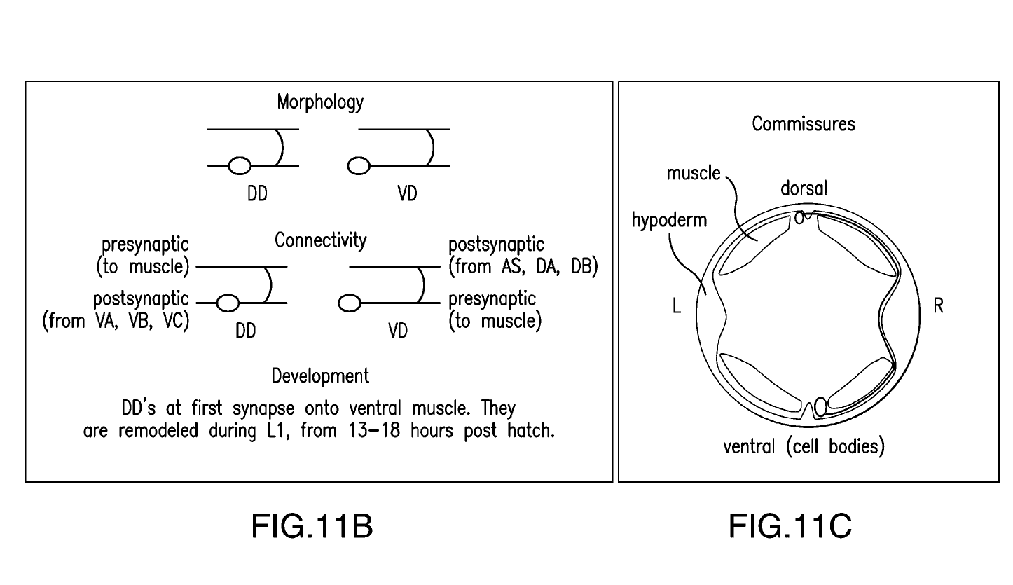

A 440 nm pulsed dye laser from Photonic Instruments was used to perform laser axotomy as described by Wu et al. (2007). Six DD motor neurons develop embryonically and 13 VD neurons develop post embryonically during the L2 stage. All but the “vulval” D neurons exhibit robust regeneration within 12 hours of laser axotomy (Wu et al. 2007). The D neurons therefore provide an excellent model system in which to assay regeneration (FIG. 11). The focus was on VD11, VD10, DDS, and VD9 because the preliminary experiments showed that cutting these axons did not seem to adversely affect worm viability or growth from L4 to adult. In all experiments, 1-4 D commissure axons were cut approximately midway between the dorsal and ventral nerve cord (red wavy line in upper panel of FIG. 11). L4 stage worms were mounted on agarose pads, anesthetized with 10 mM muscimol in M9, cover slipped, and axons were laser cut. Worms were recovered from the slide and returned to a Petri plate seeded with bacteria. Experimental worms normally recovered from the anesthetic and moved normally around the plate within 30 minutes and by the next day had developed into young adults. Worms were assayed for regeneration 14-18 hrs after axotomy.

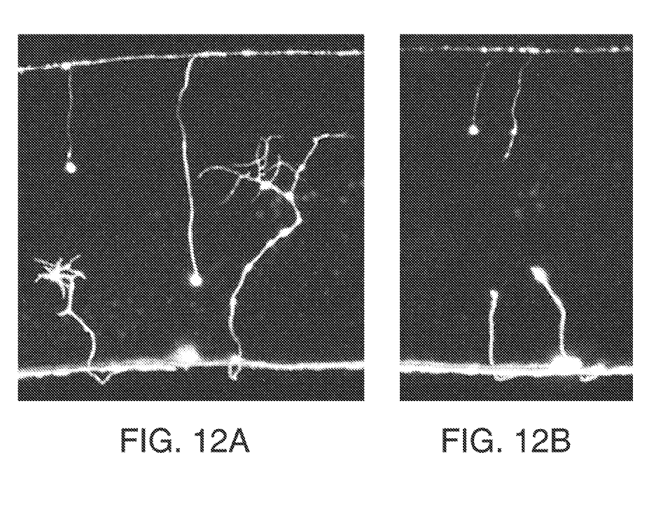

In FIG. 12 representative examples of the response to axotomy in wild type and dlk-1 D neurons are shown. In panel A the two D axon distal stumps end in retraction bulbs. Both the proximal and distal cut ends of the axons retract shortly after axotomy. After an average of 5.5 hrs, the wild type proximal stumps regenerate growth cones and begin extension towards the dorsal nerve cord. The response of the dlk-1 D neurons is completely different. The proximal stumps never regenerate a growth cone, even 5 days post axotomy (FIG. 12B).

dlk-1 has no visible developmental or adult phenotype. Its nervous system develops normally, and yet it is absolutely required for regeneration in the adult. The p38 MAPK pathway has been characterized in worms by Nakata et al. 2005 for its roll in regulating presynaptic development. The characterization of this signaling pathway was examined, and in addition asked whether other MAPK signaling pathways identified in C. elegans might play a role in neural regeneration (Sakaguchi et al. 2004). 13 genes known to be directly involved in MAPK signaling and that also offered existing mutations were observed. p-unc-47::GFP was crossed into each strain and examined the phenotype of the D motor neurons. In each case, there was either no phenotype or mild branching and pathfinding defects. D neuron axons were laser cut in L4 worms of each genotype and scored regeneration after 14-18 hrs as described above. At least 20 animals of each genotype were scored. Table 2 shows the results of these experiments. Wild type worms regenerate D neuron axons 70% of the time, similar to the frequency reported by Wu et al. (2007). However, dlk-1, mkk-4, and pmk-3 regenerate 0%. This result is consistent with the DLK-1 (MAPKKK) to MKK-4 (MAPKK) to PMK-3 (MAPK) signaling pathway. Nakata et al. (2005) also reported that RPM-1 together with FSN-1 targets DLK-1 for degradation. It was found that regeneration is somewhat improved to 86% in both the rpm-1 and fsn-1 mutant background. An over expression construct for DLK-1 (Ex[p-unc-47::dlk-1]) and RPM-1 (Ex[p-unc-25::rpm-1]) was then made to test whether the affect on regeneration was consistent with the known molecular interactions. The Ex[rpm-1] worms showed a dramatic reduction in regeneration to 7%, while the Ex[dlk-1] worms showed a significant improvement to 98%. The results for most other genotypes tested were not significant, with the provocative exceptions of jnk-1, mlk-1, mek-1 and glo-1 (Grill et al. 2007; Sakaguchi et al. 2004).

These laser axotomy experiments indicate that DLK-1 is an essential regulator of neural regeneration and mediates its affect through the pmk-3 (p38) MAPK pathway. It was then tested whether DLK-1 might act through any other pathways by testing axon regeneration in pmk-3 worms expressing the Ex[dlk-1] transgene. The phenotype in pmk-3 animals over expressing DLK-1 is identical to the pmk-3 phenotype—no regeneration of D axons. Thus, DLK-1 seems to be acting solely through the PMK-3 (p38) MAPK pathway to affect regeneration. Finally, the function of DLK-1 during neural development was tested to determine if DLK-1 enabled regeneration ability in the adult, rather than being essential at the time of regeneration. A heat shock dlk-1 construct [Phsp-16-2::dlk-1-mCherry] was made and injected it into dlk-1 worms. These L4 dlk-1 worms still do not regenerate D axons, however, heat shocking to drive expression of DLK-1 in the adult restores the D axon ability to regenerate. This supports the fact that DLK-1 function is required at or near the time of axotomy to facilitate regeneration.

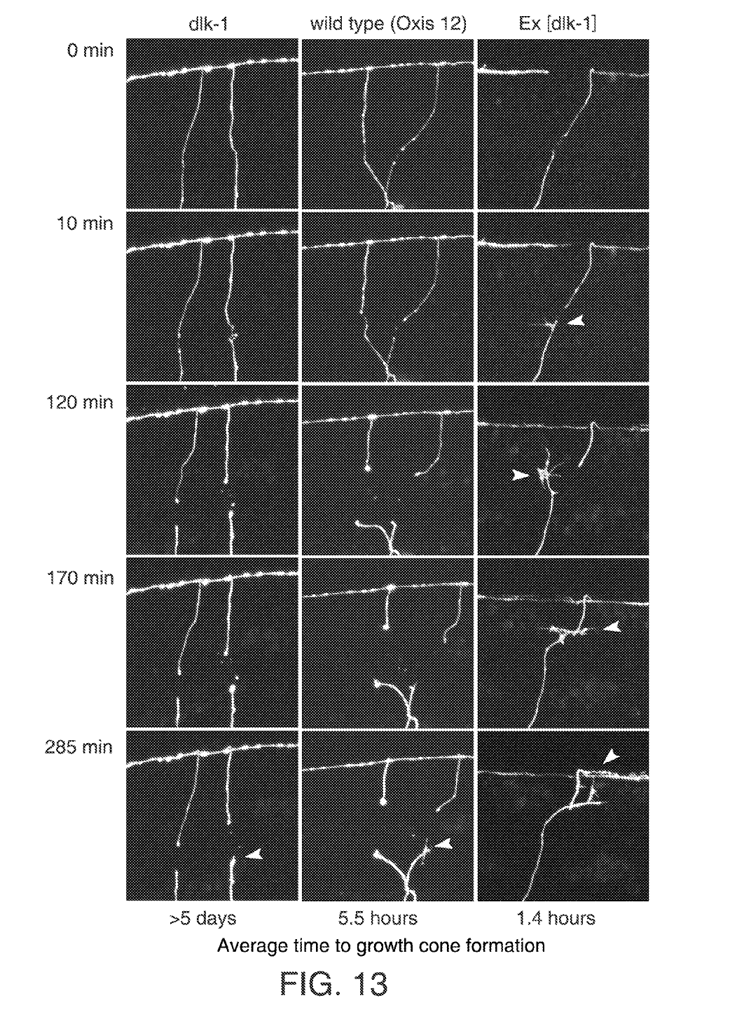

Laser axotomy data showed that over expressing DLK-1 actually improves regeneration of D axons. In wild type worms, 70% of D axons regenerate while in worms expressing the Ex[p-unc-47::dlk-1] transgene 98% of the D axons regenerate. The extent of regeneration is even more dramatic than the laser axotomy data indicate. Almost every D axon in animals over expressing DLK-1 regenerated fully back to the dorsal nerve cord. Although 70% of wild type axons extended a growth cone and began growth back to the dorsal nerve cord, fewer actually made it. Many looked as if they stalled out before reaching the dorsal nerve cord. Furthermore, time lapse studies have provided insight into the changes associated with this improvement. Time lapse recordings of regenerating axons in the dlk-1, wild type, and Ex[p-unc-47::dlk-1] animals were made. L4 worms of each genotype were anesthetized on agarose pads, D axons were laser cut as described above, and the axon imaged at 5 minute intervals for 12-20 hours using a Microradiance confocal microscope with a 60×na 1.4 objective. These time-lapse images allowed for an understanding the improved regeneration seen in neurons over expressing DLK-1. FIG. 13 shows illustrative time points from the three genotypes. The cut dlk-1 axons form retraction bulbs (120 min) similar to wild type, but never extend a growth cone or show any protrusive activity (arrowhead, 285 min). In separate experiments, cut axons in dlk-1 animals were followed up to 5 days and never observed any regeneration. In the wild type animals, by 285 min (arrowhead) only one of the cut axons begins to extend a growth cone. The average time after laser axotomy for a wild type D axon to extend a growth cone is 5.5 hours (average of 10 animal time lapse experiments). Ex[dlk-1] axons behave quite differently. In this example the D axon over expressing DLK-1 extends a growth cone only 10 minutes (arrowhead) after axotomy. The average time to form a growth cone is 1.5 hours (average of 10 animal time lapse experiments). Furthermore, the Ex[dlk-1] growth cone extends and reaches the dorsal nerve cord by 285 minutes (arrowhead), just as the wild type is first forming a growth cone. The time lapse images also show why the Ex[dlk-1] growth cones are more successful in reaching the dorsal nerve cord. It is clear that many wild type regenerating growth cones fail because they stall out when the “growth cone” transitions to a highly branched terminal structure. In contrast, the Ex[dlk-1] growth cones look and behave more like wild type embryonic DD or larval VD growth cones; they maintain their growth cone structure as they extend and do not become excessively branched and then stall out. In short, the present studies show that DLK-1 causes growth cone initiation to occur faster, with virtually 100% frequency, and growth cones are more like “embryonic” growth cones in form, behavior, and successful regeneration.

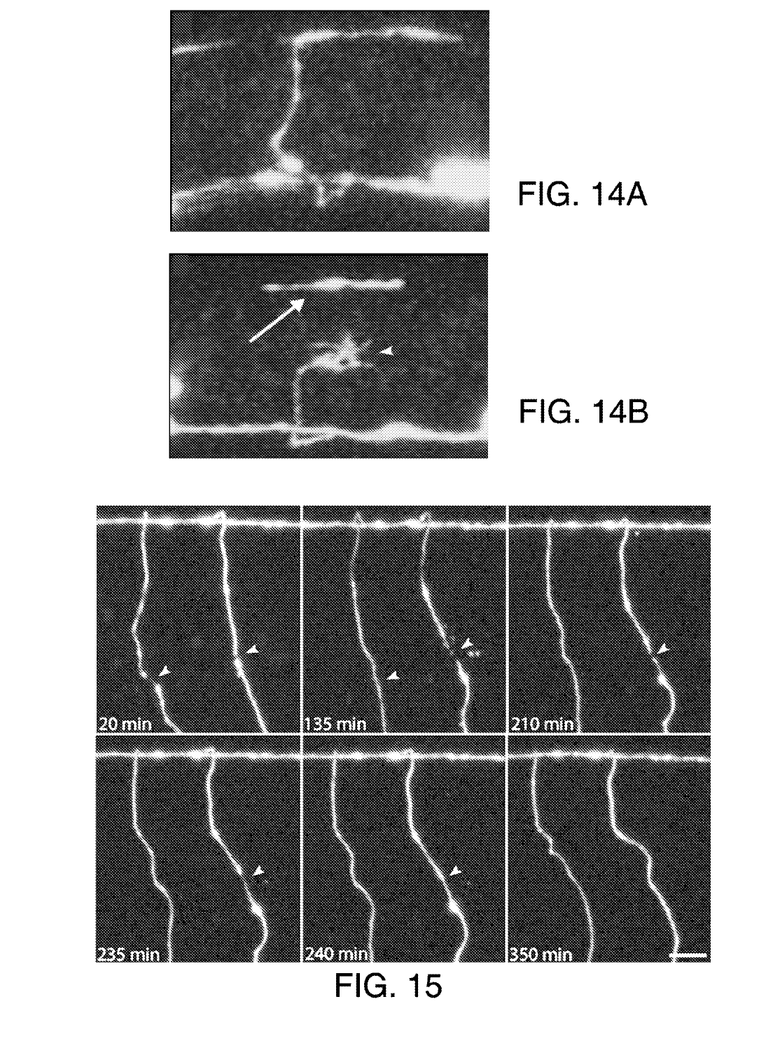

Disclosed herein is a robust screening assay for axon regeneration afforded by the characterization of the β-spectrin mutation (FIG. 9 and FIG. 14).

Screen all non-redundant clones in the Ahringer and Orfeome RNAi feeding libraries for effects on axon regeneration. All non-redundant clones in the Ahringer (Kamath et al. 2003) and Orfeome (Rual et al. 2004) RNAi feeding libraries are screened by feeding bacteria expressing each clone to β-spectrin mutant worms. Phenotypes of each clone are quantitatively scored by high powered (400×) microscope examination of identified GABAergic neurons (DD and VD motor neurons). RNAi knockdown of a gene affecting regeneration shows up as L4 worms with quantitatively fewer D neuron commissures that extend in the dorsal nerve cord, and the absence of branching and growth cones that indicate regeneration (FIG. 10).

A “prioritized” list of 2075 genes expressed only in animals with nervous systems and a secondary list of about 3410 genes that represent the remaining orthologs between C. elegans and humans has been generated (Cheng et al. 2006). To date, approximately 2354 genes have been screened and 88 positives that include transcription factors, cell surface or secreted molecules, cytoskeletal associated proteins, molecules involved in cellular metabolism, signaling proteins, and other uncharacterized proteins have been found. (This is a hit rate of about 3-4% from the orthologs list and predicts about 100-200 genes can be identified as affecting regeneration.) A very high rate of positive retests, 90% (19/21) have been found, and no negatives that retested positive.

Bacteria are grown in individual culture tubes or in 96-deep well culture plates. 350 μL of cosmid containing bacteria in log phase growth is spotted onto each RNAi plate (60 mm plates containing nutrient agar+1 mm IPTG and 25 μg/mL Carbenicillin). Plates are then kept at room temperature for 18-36 hours to let dry and undergo induction. They are stored at 4° C. for a maximum of one week before use. Control plates are seeded with empty vector (HT115/L4440) or GFP RNAi expressing bacteria. The majority of the clones are grown in 96-deep well culture plates. Cosmid containing bacteria are transferred with a 96-place pin replicator from a frozen library plate to a deep well culture plate containing 500 μL/well of LB+50 μg/mL ampicillin. This culture is grown to stationary growth phase overnight in a 37-deg. C. shaking incubator. A dilution of this culture is made by discarding 400 μL and adding fresh media. This is grown to log phase, also in a 37-deg. C. shaking incubator. (It takes about 3 hours to reach an OD600 of 0.5-0.8 because there is less gas mixing in the plate than in larger tubes or flasks.)

Worms, p-unc-47::gfp; p-unc-17::β-spectrin; p-unc-17::mCherry; unc-70; eri-1; lin-15B are grown on 100 mm plates spotted with HB101. Sterile M9 buffer is used for washing. Worms are rinsed from the HB101 plates and put over 30 μm nylon mesh, which retains gravid adults. The adult worms are washed 3× (50 ml conical tube, clinical centrifuge). Bleach solution (4 ml ddH₂O+0.5 mL bleach and 0.5 ml 10N NaOH) is added to the pelleted worms, and they are vortexed vigorously for 4-5 min. Bleaching is monitored with a dissecting scope, and as soon as the embryos are released they are spun down, washed 1× with at least 15 mL M9, refluxed gently with a 200 μL pipetter to separate the embryos, and resuspended in M9. The embryos are counted with a haemacytometer, 450 embryos are added to each RNAi plate, and the plates are kept at 15° C. until assayed.

Each batch of 50 seeded RNAi plates includes a “positive” control seeded with bacteria expressing GFP RNAi (FIG. 8b) and a “negative” control seeded with bacteria expressing the empty vector RNAi (FIGS. 8a and 9). Plates are kept at 15° C. and individually monitored for worm growth. Control plates are normally ready to screen by day 8-10. Roughly 100 worms of all ages, including at least 10 L4s, are mounted on agar pads, anesthetized with 20 mM sodium azide in M9, and cover slipped. 10 L4 worms are randomly chosen and the 19 D motor neurons are scored for GFP expression. RNAi knockdown of GFP in GABA neurons is variable and mosaic, but averages of 80-90% knockdown are consistently seen, i.e., only 1-3 D neurons per worm averaged over 10 L4 worms scored are counted. This control is reasonable since the p-unc-47 promoter drives high levels of expression of GFP in GABA neurons and it is these same GABA D neurons that are being screened for effects on regeneration. Next, the worms on the empty vector control plate are sampled. In this case the number of D commissures that reach and extend along the dorsal nerve cord in 10 L4 worms are scored. The average number per worm is consistently 9-11, although numbers as high as 15 and as low as 5 in single worms have been observed. If both the positive and negative controls are satisfactory then the experimental plates are screened in the same way by quantifying the D commissures in 10 L4 worms. Averages of less than 7 are scored as positives. All initial positives are retested on RNAi plates and if they score positive again are deemed “real” and prioritized based on existing alleles and a molecular informatics rationale.

All clones that cannot be scored in the first round because of sterility or lethality are screened by screening the parental generation for phenotypes. Maintenance defects in worms can be seen (after 5-7 days) with some control genes, e.g., unc-70 and unc-119. The subset of sterile/lethal clones are screened in a genetic background that limits RNAi knockdown to GABA neurons. Unc-70; p-unc-47::gfp; p-unc-17::β-spectrin; p-unc-17::mCherry; p-unc-47::rde-1; eri-1; lin-15 B; rde-1 are screened this way to make all cells, except the GABA neurons, insensitive to the effects of RNAi (Qadota et al. 2007). If these worms are viable that allows for regeneration defects specific to cell intrinsic protein function in GABA neurons to be scored. This strain is used to retest clones that are viable, but score as “improved” regeneration (less than 3%). It has been found that paralyzing worms rescues the β-spectrin phenotype simply by preventing the movement induced axon breakage (Hammarlund et al. 2007). GABA neuron specific RNAi allows for the assay of the effect on regeneration separate from the confounding effects of animal paralysis.

Validate and characterize all “positives” identified in the RNAi screen by laser axotomy of GABA neuron axons in genetic mutations. GABA neuron GFP marker are crossed into existing genetic mutations for genes identified in the RNAi screen. Any developmental defects in D axon commissures are compared with the regeneration phenotype after laser axotomy to judge the relative role-played by each gene in axon regeneration. The ultimate test of each candidate protein is to over “activate” or “inhibit” its function in regenerating neurons (or the environment) and assay its affect on improving regeneration.

Worms of each candidate genotype are grown from synchronized egg plates and scored for developmental defects. 100 L4 worms are mounted on an agarose pad, anesthetized with 20 mM sodium azide, cover slipped and viewed at 400×. GABA neurons are scored for any defects, i.e., branches, pathfinding errors, gaps in dorsal and ventral nerve cord, missing commissures or cell bodies or aberrant growth cones. Next, 1-3 L4 worms are mounted on a agarose pad, anesthetized with 10 mM muscimol, cover slipped and viewed with a 60× planApo 1.4 na lens. One to four D axon commissures in each worm are targeted for laser axotomy (FIG. 11) using a Photonic Instruments Micropoint 440 nm laser. After successful laser axotomy the worms are recovered to fresh Petri plates seeded with HB101 and kept at 20 degrees C. 14-20 hours later the worms are evaluated for normal maturation to fertile young adults and if deemed healthy they are again mounted on agarose pads, paralyzed and imaged with a laser scanning confocal microscope as illustrated in FIG. 12. A minimum of 20 animals (and 40 axons) of each genotype are scored for regeneration after laser axotomy (see Table 2 as an example data set). Commissural D axons are cut at the midpoint between the ventral nerve cord and the dorsal nerve cord. The results of the laser axotomy experiments are evaluated, together with other criteria, to prioritize candidates for further analysis.

Characterize the dynamic phenotypes of neural regeneration in key neuron cell types. The regeneration phenotype is complex and needs to be fully characterized in terms of regeneration in representative neuron cell types to create quantitative assays for the analysis of gene function during regeneration. Motor, sensory, and interneurons can respond differently to axotomy, as well as regional differences in dendritic and axonal regeneration (Wu et al. 2007; Gabel et al. 2008). Age related changes can also be quantified (Wu et al. 2007). In addition to reproducing some of the work of Wu et al. (2007) and Gabel et al. (2008) relatively novel phenotypes can be characterized. FIG. 15 shows two axons that are laser “cut” in preparation of a time-lapse experiment. At 20 minutes both axons had retraction bulbs and looked fully separated from their distal segments (arrowheads). However, a faint “wisp” of GFP labeling between the proximal and distal ends of the left axon can be seen. At 135 min the left hand axon has fully repaired itself, while the right axon still seems cut from its distal segment (arrowheads). Note the punctate GFP debris near the cut site (arrowhead left axon, 135 min). At 210 minutes, the right axon has a well-formed retraction bulb, but seems to have “extended” a thin tube towards the distal stump. By 240 minutes, the proximal tube-like extension contacts the distal fragment and by 350 minutes the repair is complete and both commissural axons look undamaged. It appears that the laser damage to the axon breaks down the axon cytoskeleton that supports the axonal membrane, but leaves a thin cytoplasm free tube of membrane lipid intact. As long as this tube remains there is a possibility of “repair”. Repair is seen along the same tract as the original axon, even in the face of laser damage to the local environment. The repair always occurs without the appearance of a growth cone. Repair has never been seen when a regenerating growth cone contacts its own distal segment. The repair appears to represent true cytoplasmic continuity because in time-lapse movies the engorged proximal “stump” cytoplasm quickly flows down the axon into the distal segment across the repaired region, e.g., note the swollen “retraction bulb” disappears between 240 and 350 minutes (FIG. 15). A “repair” phenotype has been shown to be relevant to recovery after acute crushing trauma of the spinal cord in mammals (Rossingnol et al. 2007; Kerschensteiner et al. 2005). No one has an in vivo assay with single cell resolution for this repair behavior in a genetically tractable model system. It has also been shown that the repair process is under the genetic control of some genes involved in neural regeneration.

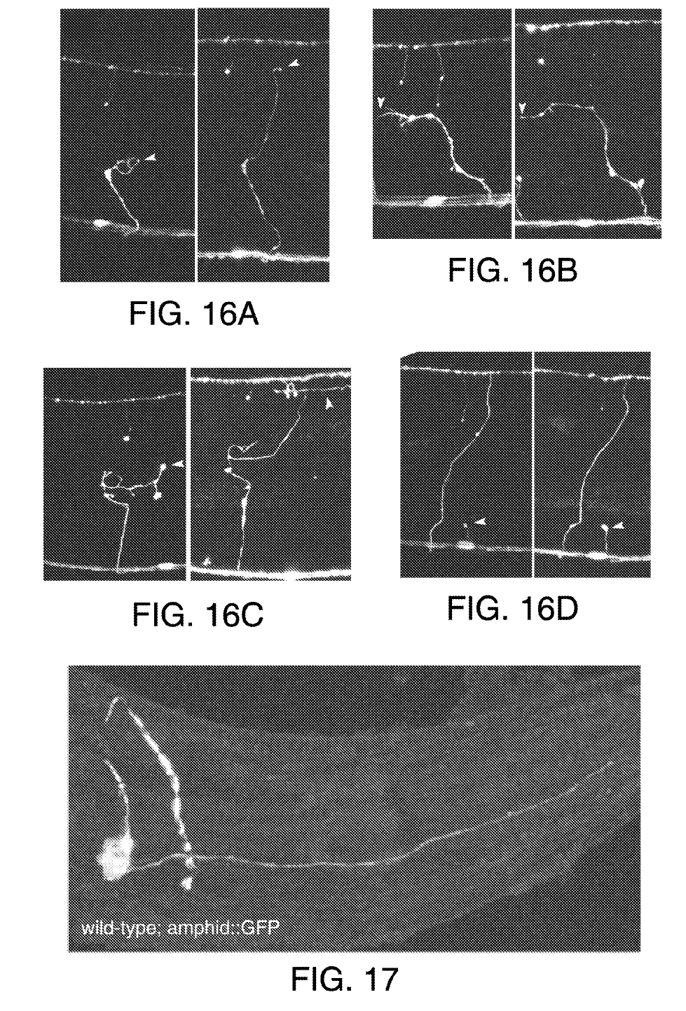

D axon regeneration is fairly robust after 12-24 hours (Wu et al. 2007), but even after 5 days some wild type D axons never successfully regenerate back to the dorsal nerve cord (FIG. 16). D axon commissures were cut in ten animals and imaged after 24 hours. Axons in 4 animals had not successfully regenerated back to the dorsal nerve cord. These animals from the slide and returned them to Petri plates with food for 4 more days. At the end of this time they all appeared healthy and the D axons were re-imaged. It was found that if axons do not mount a successful regeneration response within 24 hours, then even after 5 days they generally do not succeed (FIG. 16). In A significant growth is observed, but no active looking growth cone and the axon stopped short of the dorsal nerve cord. In B several branches retract and no significant new growth is seen. In C the axon looks like it might have made it back to the dorsal nerve cord. D is the most severe case where no growth cone or branching was ever seen, yet the axon stump looks “healthy” even after 5 days. This is an important phenotype to understand because ultimately it can explain why wild type axons fail to regenerate, and just as importantly if these “old” axons stumps can be induced to extend growth cones. The clinical relevance of this is that there are many people living with spinal cord damage that occurred years ago and if “senescent” axon stumps can be induced to regenerate, then they can be treated that way. It has been demonstrated that over expressing DLK-1 actually improves several metrics of regeneration in D motor neurons. Preliminary results have been found showing that the dlk-1 “non-regenerating” phenotype can be rescued in the adult by expressing DLK-1 from a heat shock construct [Phsp-16-2::dlk-1-mCherry]. Once the heat shock protocol and phenotype are carried out, one can identify wild type “non-regenerating” axons as shown in FIG. 16(A,B,D), and it can be determined if regeneration can be turned back on in these failed axons by expressing high levels of DLK-1. Similar types of experiments can be done on “old” animals in which regeneration frequency and success is dramatically reduced.

Can regeneration be turned on in non-regenerating neurons? Some sensory neuron (Amphids and Phasmids) dendrites normally do not mount a regeneration response after laser axotomy (Chung et al. 2006; Yanik et al. 2004; Wu et al. 2007; Gabel et al. 2008). Different cell specific promoters can drive high levels of GFP and candidate protein expression in “non-regenerating” neurons. An example can be seen in FIG. 17 that illustrates the amphid neuron ASI with a clearly identifiable dendrite extending anteriorly to the “nose” and an axonal process circling in the nerve ring. The regeneration response of the ASI axon and dendrite are examined by laser axotomy.

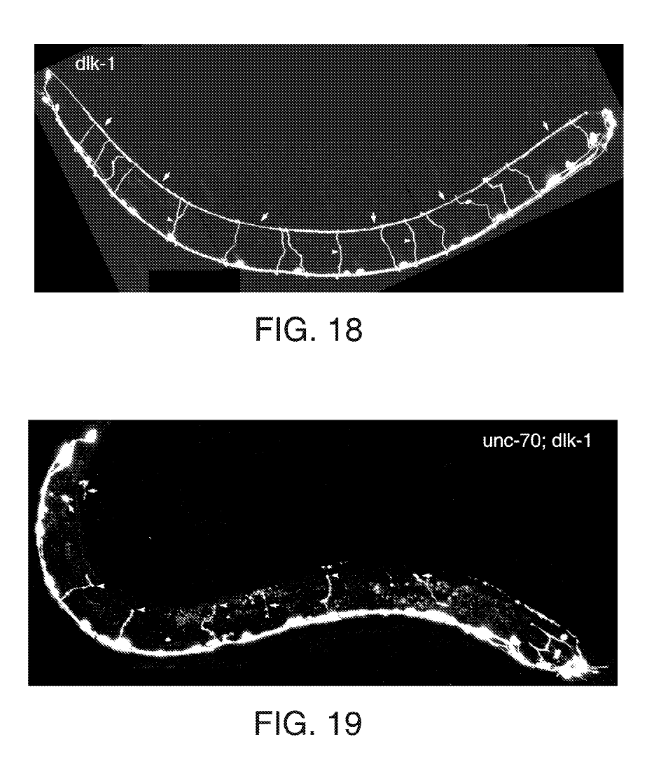

In depth analysis of key regulatory pathways controlling axon regeneration. The criteria for choosing genes for in depth analysis: (1) Strength and characteristics of the phenotype; (2) Relative specificity for regeneration vs. development; (3) Relative specificity for neurons versus other cell types; (4) Molecular “rationale” based on known molecular functions. Using these criteria the in depth analysis of the PMK-3 (p38 MAPK) pathway controlled by DLK-1 was carried out as a key regulator of axon regeneration. In the primary and secondary screen of dlk-1 RNAi in the unc-70 background the regeneration phenotype was highly “penetrant” (score<5 commissures to dorsal nerve cord). In addition, there were no growth cones or aberrant branches to indicate any regeneration had taken place (FIG. 10). Published reports suggested dlk-1 played a role in pre-synaptic development, but was not obviously required for “normal” development of the nervous system (Nakata et al. 2005). The behavior of dlk-1; p-unc-47::gfp worms is superficially normal and the GABA nervous system has wild type commissures (arrowheads) and a complete dorsal nerve cord (arrows) (FIG. 18). However, the dlk-1; unc-70 double mutant shows exactly the phenotype predicted based on the results of the RNAi screen phenotype (FIG. 19). The DD and VD axons grow out normally during development and the L2 stage, but the movement induced axon breaks cannot be rescued by neural regeneration. The dorsal nerve cord is entirely missing, and you can see D axon stumps (arrowheads) and distal axon fragments (arrows) (FIG. 19). Note that just as in the RNAi experiment (FIG. 10), there are no obvious regenerating growth cones or regenerative branches. Next the regeneration phenotype was characterized by laser axotomy in the dlk-1; p-unc47::gfp strain as described in FIG. 12. In laser axotomy experiments on over 24 animals and 69 D axons regeneration response has never been observed in animals lacking functional DLK-1 (Table 2). DLK-1 functions in the MKK-4 to PMK-3 (p38) MAPK signaling pathway to regulate presynaptic development in C. elegans (Nakata et al. 2005). The p38 MAPK pathway has been linked to many different functions in the nervous system and offers a rich source of candidate genes that can be involved in neural regeneration (Takeda et al. 2002, Gallo et al. 2002; Sakaguchi et al. 2004; Kyosseva 2004). The core elements of the pathway were first tested as defined by Nakata et al. (2005) and in addition included other members of characterized MAPK cascades in C. elegans as a test of specificity (Nakata et al. 2005; Sakaguchi et al. 2004). Table 2 shows the results of these experiments and demonstrates the profound affect of the DLK-1 (MAPKKK) to MKK-4 (MAPKK) to PMK-3 (MAPK) pathway on regeneration. In each case there is a complete block to neural regeneration, even though the GABA nervous system in each of these mutant strains (dlk-1, mkk-4, and pmk-3) is relatively normal. The results of axotomy in the rpm-1 (86%) and fsn-1 (86%) mutants are consistent with Nakata et al. (2005) and Wu et al. (2007) results demonstrating that RPM-1/Wallenda, together with FSN-1, negatively regulates DLK-1 by targeting it for degradation. Over expressing RPM-1 (Ex[rpm-1]) strongly suppresses regeneration (7%). However, over expressing DLK-1 (Ex[dlk-1] actually seems to improve regeneration to 98%.

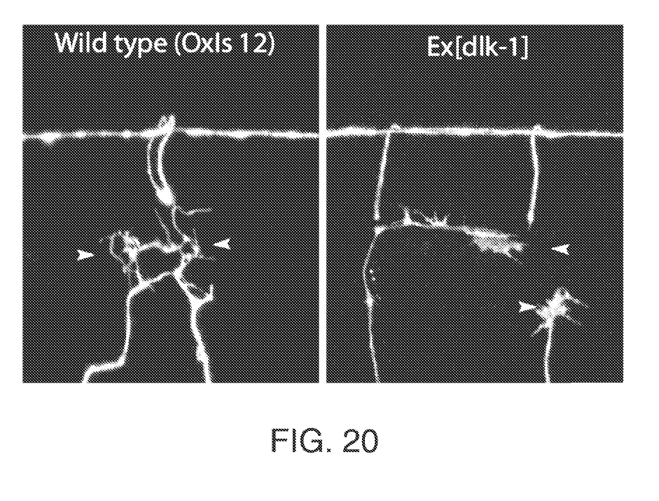

How does over expressing DLK-1 improve regeneration? DLK-1 is a member of the duel leucine zipper kinase family of MAPKKK mixed lineage kinases (Gallo et al. 2002). There is evidence that DLK homodimerizes and autophosphorylates to activate its kinase activity, so it is reasonable to expect over expression to increase signaling through the MKK-4 to PMK-3 pathway (Daviau et al. 2006). It has been shown that the effect of DLK on regeneration is solely through PMK-3 by assaying the regeneration phenotype in the Ex[dlk-1]; pmk-3 strain. There is no regeneration (preliminary data, n=10 animals). What phenotypes explain the improved regeneration of D axons over expressing DLK-1 compared to the wild type D axons? Time-lapse movies were made of dlk-1, wild type, and Ex[dlk-1] regenerating axons to understand how their regeneration phenotypes differed (FIG. 13). The selected time points shown in FIG. 13 illustrate the differences. Wild type axons that are cut by laser axotomy initiate a regenerating growth cone about 70% of the time compared to 0% for dlk-1 and 98% for Ex[dlk-1]. More importantly, wild type axons regenerate a growth cone after an average of 5.5 hours (n=10 animals), while Ex[dlk-1] axons regenerate a growth cone after an average of only 1.4 hours (n=10 animals). FIG. 13 shows an example where a Ex[dlk-1] axon regenerates a growth cone within 10 minutes of axotomy and extends successfully to the dorsal nerve cord within 285 minutes, while the wild type axon has just begun to regenerate a growth cone at 285 minutes. These time-lapse movies gave another insight into the improved behavior of growth cones over expressing DLK-1. It was observed that wild type regenerating growth cones often were not successful in regenerating to the dorsal nerve cord because they became hyper-branched and stalled out (FIG. 20). However, the appearance and behavior of the growth cones over expressing DLK-1 was more like wild type developing VD growth cones (FIG. 20) (Knobel et al. 1999; Knobel et al. 2001; Weinkove et al. 2008). The growth cones over expressing DLK-1 maintain a coherent and recognizable core as they send out filopodia and extend towards the dorsal nerve cord (FIG. 20).

In summary, dlk-1 was identified in an RNAi screen as a gene absolutely required for regeneration, but not development, of D neurons. Laser axotomy and time-lapse experiments have demonstrated the requirement for the core DLK-1 to MKK-4 to PMK-3 MAPK pathway and the apparent regulation of DLK-1 activity by RPM-1 and FSN-1 (Nakata et al. 2005). “Improved” regeneration in neurons over-expressing DLK-1 has been observed, and the growth cone behaviors that correlate with this improvement have also been shown.

In addition to RPM-1 and FSN-1, there is evidence that several phosphatases (PP1, PP2a, and calcineurin) can regulate DLK activity (Mata et al. 1996). There is also evidence that Hsp70 and the CHIP ubiquitin ligase together target active DLK for proteosome dependent degradation (Daviau et al. 2006). The targets of p38 signaling are less well understood, but there are intriguing results from Campbell et al. (2003) and Verma et al. (2005) that strongly implicate p38 signaling in neuronal chemotropic response and axonal regeneration. Their results indicate that p38 can be activated by netrin-1 and acts by phosphorylation of the eukaryotic initiation factor 4E (elf-4E) and its repressor binding protein (elf-4EBP1) to stimulate local protein synthesis (Campbell et al. 2003). Inhibiting p38 signaling by SB203580 (Tong et al. 1997) reduces regeneration in vitro by adult DRG neurons from about 65% to 10%. Inhibiting p38 not only reduces localized axonal protein synthesis, but also the proteosome mediated protein degradation seen in response to axotomy (Verma et al. 2005). Recently another target of p38 signaling has been identified, GSK-3β, that also affects localized protein synthesis. GSK-313 phosphorylates and inactivates elf-2b, so inactivation of GSK-3β by p38 stimulates an elf-2b mediated increase in translation (Thornton et al. 2008). The parallels between the p38 MAPK pathway in C. elegans and in vertebrates support the conservation of the molecular signaling pathway controlling neural regeneration. Lewcock et al. (2007) identified Phrl, the mammalian homolog of the C. elegans RPM-1, in a forward genetic screen for mutations affecting motor neuron pathfinding. The spinal motor phenotype was 100% penetrant, yet overall the nervous system must have been fairly normal because the mutant animals were not distinguished from their litter mate controls (Lewcock et al. 2007). Neuronal branching and “over growth” phenotypes in otherwise normal worms expressing Ex[dlk-1] has been observed. They localize Phr-1 to stable axonal microtubules and DLK to growth cones. The Magellan (Phr-1) phenotype is described as an expanded region of disorganized microtubules extending from the axon into the growth cone (Lewcock et al. 2007). Both Taxol and inhibitors of p38 (Tong et al. 1997) rescued this growth cone phenotype. This model is that Phr-1 restricts DLK to the growth cone by targeting it for degradation in the axon and suggests that p38 can function in this pathway by regulating the MAP doublecortin at the transitional region from axon to growth cone. Spinophilin, PP1, and doublecortin function together to bundle and stabilize microtubules and actin in axons (Bielas et al. 2007) and p38 mediated phosphorylation of doublecortin can inactivate it in the growth cone to facilitate the formation of dynamic MT associated with growth cone motility (Lewcock et al. 2007).

The working model is that DLK activity is held in check in adult neurons by RPM-1/Highwire/Phr-1 (Nakata et al. 2005; Wu et al. 2005; Fulga et al. 2008). Cutting axons removes RPM-1 activity and allows DLK-1 activity to build up in the axon stump where the activated PMK-3 (p38) can phosphorylate target proteins to destabilize the bundled axonal MT and stimulate localized protein synthesis (and degradation) to initiate growth cone formation. The model shows that the DLK-1 pathway supporting growth cone formation and extension in regeneration is parallel to the molecular pathway controlling neural development. DLK-1 and the PMK-3 (p38) MAPK pathway is required for regeneration, but not essential for development of the nervous system. In the absence of DLK-1, regeneration cannot take place because the stable axonal stump MT cannot be converted to the dynamic MT required for growth cone formation. Too little DLK-1 might result in stable MT prematurely invading growth cone exploratory branches, resulting in regeneration failure from stalled hyper-branched axons. Too much DLK-1 can cause excessive destabilization of axonal MT leading to aberrant branching and “overgrowth”.

It has been demonstrated that RNAi based screen for genes affecting neural regeneration in C. elegans works. 2354 genes have been screened from the prioritized list and 88 genes have been identified that affect regeneration. DLK-1 has been identified as a regulator of neural regeneration in the screen, and it has been demonstrated that a molecular pathway required for regeneration, but not development (DLK-1 to MKK-4 to PMK-3) can be characterized. Laser axotomy and time-lapse microscopy assays have been carried out that allow for the characterization of the cellular phenotypes associated with genes affecting neural regeneration. The core RNAi screening, laser axotomy, and time-lapse imaging techniques are all used successfully. Disclosed herein is a unique axotomy-regeneration phenotype in the C. elegans β-spectrin mutant that makes possible an “unbiased” screen for genes effecting neural regeneration. The identification of all genes effecting the regeneration of neurons provides for a rational targeted approach to developing new therapeutic agents (Horner et al. 2000; Case et al. 2005; Du et al. 2007; Kubo et al. 2007).

Disclosed herein are methods of regenerating axons. Disclosed herein are also methods of regenerating axons in a subject, which includes but is not limited to mammals such as human beings.

In one aspect, the methods of regenerating axons comprise activating a gene in the p38 MAPK pathway of C. elegans. In an aspect, the methods of regenerating axons comprise activating a gene with 80% or greater homology to a gene in the p38 MAPK pathway of C. elegans. The p38 MAPK pathway includes but is not limited to the following genes: dlk-1, mkk-4, and pmk-3.

In one aspect, the methods of regenerating axons comprise inhibiting a gene in the p38 MAPK pathway of C. elegans. In an aspect, the methods of regenerating axons in a subject comprise inhibiting a gene with 80% or greater homology to a gene in the p38 MAPK pathway of C. elegans. The p38 MAPK pathway includes but is not limited to the following genes: rpm-1, fsn-1, and phr-1.

The disclosed methods of regenerating axons can use siRNA to inhibit a gene in the p38 MAPK pathway of C. elegans or to inhibit a gene with 80% or greater homology to a gene in the p38 MAPK pathway of C. elegans.

Disclosed herein are methods of treating a subject with a neurodegenerative disease. In an aspect, the methods of treating a subject with a neurodegenerative disease comprise activating a gene with 80% or greater homology to a gene in the p38 MAPK pathway of C. elegans. In an aspect, the methods of treating a subject with a neurodegenerative disease comprise inhibiting a gene with 80% or greater homology to a gene in the p38 MAPK pathway of C. elegans.

Disclosed herein are methods of regenerating axons in a subject. In an aspect, the methods of regenerating axons in a subject comprise activating a gene with 80% or greater homology to a gene in the p38 MAPK pathway of C. elegans. The disclosed methods further comprise contacting a gene in the p38 MAPK pathway with a compound that activates said gene.

Disclosed herein are methods of regenerating axons in a subject. In an aspect, the methods of regenerating axons in a subject comprise inhibiting a gene with 80% or greater homology to a gene in the p38 MAPK pathway of C. elegans. The disclosed methods further comprise contacting the gene in the p38 MAPK pathway with a compound that inhibits said gene.

Disclosed herein are pharmaceutical compositions. In an aspect, the disclosed pharmaceutical compounds can be used in the disclosed methods of regenerating axons. In an aspect, the disclosed pharmaceutical compositions activate a gene with 80% or greater homology to a gene in the p38 MAPK pathway. In an aspect, the disclosed pharmaceutical compositions inhibit a gene with 80% or greater homology to a gene in the p38 MAPK pathway.

Disclosed herein are methods of screening for test compounds that modulate the p38 MAPK pathway of C. elegans. The disclosed methods of screening for test compounds that modulate the p38 MAPK pathway comprise contacting a gene in the p38 MAPK pathway with a test compound and detecting interaction between the gene and the test compound. In the disclosed methods, interaction between the gene and the test compound indicates a test compound that modulates the p38 MAPK pathway. In an aspect, the modulation of the p38 MAPK pathway comprises activation of a gene in the p38 MAPK pathway. In an aspect, the modulation of the p38 MAPK pathway comprises activation of a gene with 80% or greater homology to a gene in the p38 MAPK pathway. The p38 MAPK pathway includes but is not limited to the following genes: dlk-1, mkk-4, and pmk-3.

Disclosed herein are methods of screening for test compounds that modulate the p38 MAPK pathway of C. elegans. The disclosed methods of screening for test compounds that modulate the p38 MAPK pathway comprise contacting a gene in the p38 MAPK pathway with a test compound and detecting interaction between the gene and the test compound. In the disclosed methods, interaction between the gene and the test compound indicates a test compound that modulates the p38 MAPK pathway. In an aspect, the modulation of the p38 MAPK pathway comprises inhibition of a gene in the p38 MAPK pathway. In an aspect, the modulation of the p38 MAPK pathway comprises inhibition of a gene with 80% or greater homology to a gene in the p38 MAPK pathway. The p38 MAPK pathway includes but is not limited to the following genes: rpm-1, fsn-1, and phr-1.

In an aspect, the disclosed methods of screening for test compounds that modulate the p38 MAPK pathway of C. elegans further comprise contacting the test compound with one or more axons.

Disclosed herein are methods of screening for test compounds that modulate the p38 MAPK pathway, wherein the test compound or a plurality of test compounds are contacted with a gene in the p38 MAPK pathway in a high throughput assay system. In an aspect, the high throughput assay system comprises an immobilized array of test compounds. In an aspect, the high throughput assay system comprises an immobilized array of a gene in the p38 MAPK pathway.

Disclosed herein are compounds that are identified by methods of screening for test compounds that modulate the p38 MAPK pathway of C. elegans, wherein modulation comprises activation of a gene in the p38 MAPK pathway or a gene with 80% or greater homology to a gene in the p38 MAPK pathway.

Disclosed herein are compounds that are identified by methods of screening for test compounds that modulate the p38 MAPK pathway of C. elegans, wherein modulation comprises inhibition of a gene in the p38 MAPK pathway or a gene with 80% or greater homology to a gene in the p38 MAPK pathway.

TABLE 1
Listing of the Strains and Complete Genotypes

| | | | # | # | | P vs. |
|---|---|---|---|---|---|---|
| | Genotype | Strain | animals | axons | # regen. | control |
| wild type | oxls12 | EG1285 | 50 | 105 | 73 | |
| MAPKKK | dlk-1(ju476); oxls12 | MJB1014 | 24 | 69 | 0 | <.0001 |
| vDLK-1 | dlk-1(ju476) basEx2 | MJB1032 | 21 | 53 | 43 | 0.1314 |
| no HS | dlk-1(ju476); oxls12 | EG5203 | 22 | 55 | 0 | <.0001 |
| HS 0 | ″ | ″ | 13 | 25 | 21 | 0.2135 |
| HS +2 | oxls12 | EG1285 | 20 | 51 | 35 | 1 |
| HS −2 | dlk-1(ju476); oxls12 | MJB1014 | 19 | 46 | 0 | <.0001 |
| HS−11 | dlk-1(ju476); oxls12 | EG5203 | 3 | 9 | 0 | <.0001 |
| HS −8 | ″ | ″ | 15 | 37 | 2 (5%) | <.0001 |
| HS −4 | ″ | ″ | 10 | 23 | 8 (35%) | 0.0034 |
| HS −2 | ″ | ″ | 7 | 17 | 7 (41%) | 0.0292 |
| HS 0 | ″ | ″ | 13 | 25 | 21 | 0.2135 |
| HS +2 | ″ | ″ | 10 | 24 | 10 | 0.0169 |
| HS +4 | ″ | ″ | 17 | 46 | 23 | 0.0276 |
| HS +8 | ″ | ″ | 14 | 34 | 13 | 0.002 |
| HS +24 | ″ | ″ | 22 | 60 | 8 (13%) | <.0001 |
| HS +48 | ″ | ″ | 35 | 96 | 1 (1%) | <.0001 |
| MAPKK | mkk-4(ju91); | MJB1015 | 31 | 76 | 0 | <.0001 |
| MAPK | pmk-3(ok169); | MJB1013 | 22 | 69 | 0 | <.0001 |
| MAPKKK | nsy-1(ok593); oxls12 | MJB1026 | 21 | 48 | 34 | 1 |
| MAPKK | sek-1(km4); oxls268 | MJB1022 | 23 | 51 | 28 | 0.0777 |
| MAPKK | jkk-1(km2); oxls268 | MJB1021 | 24 | 46 | 27 | 0.2618 |
| MAPKKK | mlk-1(ok2471); | MJB1029 | 24 | 52 | 12 | <.0001 |
| MAPKK | mek-1(ks54); | MJB1020 | 21 | 46 | 22 | 0.0168 |
| MAPK | jnk-1(gk7); oxls12 | MJB1023 | 27 | 68 | 65 | <.0001 |
| DLK-1 OE | basEx1; oxls268 | MJB1011 | 22 | 43 | 42 | <.0001 |
| ″ | mkk-4(ju91); | MJB1038 | 24 | 50 | 14 | <.0001 |
| ″ | pmk-3(ok169); | MJB1039 | 25 | 46 | 3 (7%) | <.0001 |
| RPM-1 OE | basEx3; oxls268 | MJB1034 | 22 | 43 | 3 (7%) | <.0001 |
| E3 ligase | rpm-1(ju41); oxls12 | MJB1027 | 24 | 51 | 44 | 0.0296 |
| F-Box | fsn-1(gk429); oxls12 | MJB1025 | 24 | 65 | 56 | 0.0163 |
| Rab GTPase | glo-1(zu391); | MJB1017 | 24 | 65 | 33 | 0.0155 |
| Rab GEF | glo-4(ok623); oxls12 | MJB1024 | 25 | 59 | 39 | 0.7272 |

| Transgenes | Allele | Contents |
|---|---|---|
| GFP | oxls12 X | Punc-47:GFP, lin-15(+) |
| GFP | oxls268 III | Punc-47:GFP |
| DLK-1 rescue | BasEx2 | Punc-47:DLK-1 cDNA-GFP; Punc-47:mCherry; Pmyo- |
| DLK-1 HS | oxEx1268 | Phsp-16.2:DLK-1 cDNA, Pmyo-2:GFP |
| DLK-1 OE | BasEx1 | Punc-47:DLK-1 minigene, Pmyo-2:mCherry |
| RPM-1 OE | BasEx3 | Punc-25:RPM-1 genomic (pCZ480—gift of Yishi Jin), |

TABLE 2
MAP Kinase Pathways Tested by Laser Axotomy in C. elegans.

| Protein | Gene | % regeneration^{a} | % sprouting^{b} | % no regeneration | n commisures ^{c} | n animals^{d} | p^{e} |
|---|---|---|---|---|---|---|---|
| | wild-type | 70 | 17 | 13 | 105 | 50 | |
| MAPKKK | dlk-1 | 0 | 1 | 99 | 69 | 24 | <.0001 |
| MAPKKK | mlk-1 | 23 | 23 | 54 | 52 | 24 | <.0001 |
| MAPKKK | nsy-1 | 71 | 15 | 15 | 48 | 21 | 0.8825 |
| MAPKK | mkk-4 | 0 | 3 | 97 | 76 | 31 | <.0001 |
| MAPKK | sek-1 | 56 | 27 | 18 | 51 | 23 | 0.0659 |
| MAPKK | jkk-1 | 59 | 22 | 20 | 46 | 24 | 0.2618 |
| MAPKK | mek-1 | 48 | 30 | 22 | 46 | 21 | 0.0058 |
| MAPK | pmk-3 | 0 | 3 | 97 | 69 | 22 | <.0001 |
| MAPK | jnk-1 | 96 | 3 | 1 | 68 | 27 | <.0001 |
| | Ex[dlk-1+++]^{f} | 98 | 2 | 0 | 43 | 22 | <.0001 |
| E3 ligase | rpm-1 | 86 | 6 | 8 | 51 | 24 | 0.0305 |
| | Ex[rpm-1+++]^{g} | 7 | 12 | 81 | 43 | 22 | <.0001 |
| F-Box | fsn-1 | 86 | 9 | 5 | 65 | 24 | 0.0132 |
| Rab GEF | glo-4 | 66 | 15 | 19 | 59 | 25 | 0.4795 |
| Rab GTPase | glo-1 | 51 | 14 | 35 | 65 | 24 | <.0001 |

^{a}percent commisures with growth cones present on proximal fragment and/or net growth of 5 μm or more
^{b}percent commisures with small branches present on proximal fragment
^{c}number of severed commisures scored
^{d}number of axotomized individuals
^{e}p value by chi square test
^{f}wild-type animals carrying an extrachromosomal array overexpressing dlk-1
^{g}wild-type animals carrying an extrachromosomal array overexpressing rpm-1

TABLE 3
Axonal Regeneration of Various MAP Kinase Pathways

| | Mammalian | | | | |
|---|---|---|---|---|---|
| | protein family | C. elegans gene | allele | % regeneration | P |
| MAPKKK | MEKK | mtk-1 | ok1382 | | |
| | | Y53F4B.1 | km43 | | |
| | ASK | nsy-1 | ok593 | 71 | 0.8825 |
| | MLK | mlk-1 | ok2471 | 23 | <.0001 |
| | DLK | dlk-1 | ju476 | 0 | <.0001 |
| | ZAK | zak-1 | km27 | 62 | 0.3777 |
| | TAK | mom-4 | gk563 | embryonic lethal | |
| | | Y105C5A.24 | km39 | | |
| | TAO | kin-18 | ok395 | | |
| MAPKK | MKK3/6 | sek-1 | km4 | 55 | 0.0659 |
| | | sek-4 | km42 | | |
| | | sek-5 | tm4028 | | |
| | MKK4 | mkk-4 | ju91 | 0 | <.0001 |
| | | sek-3 | ok1276 | | |
| | | sek-6 | ok1386 | 86 | 0.0398 |
| | MKK7 | jkk-1 | km2 | 59 | 0.2618 |
| | | mek-1 | ks51 | 48 | 0.0058 |
| | MKK5 | E02D9.1 | tm4000 | | |
| MAPK | JNK | jnk-1 | gk7 | 96 | <.0001 |
| | | kgb-1 | um3 | 3 | <.0001 |
| | | kgb-2 | gk361 | 86 | 0.0144 |
| | | Y51B9A.9 | niDf177 | | |
| | | C49C3.10 | tm3933 | | |
| | p38 | pmk-1 | km25 | 67 | 0.7455 |
| | | pmk-2 | gk21 | L1 arrest | |
| | | pmk-3 | ok169 | 0 | <.0001 |
| interacting | JSAP | unc-16 | e109 | 93 | 0.0041 |
| | JIP | jip-1 | km18 | | |
| regulatory | Phr1 | rpm-1 | ju41 | 86 | 0.0305 |
| | MKP7 | vhp-1 | km20 | | |
| | | vhp-1 | sa366 | 90 | <.0001 |

What is claimed is:

1. A method of regenerating axons, the method comprising activating a gene in the p38 MAPK pathway of C. elegans.

2. A method of regenerating axons in a subject, the method comprising activating a gene with 80% or greater homology to a gene in the p38 MAPK pathway of C. elegans.

3. The method of claim 2, wherein the subject is a mammal.

4. The method of claim 3, wherein the mammal is a human.

5. The method of claim 1 or 2, wherein the gene in the p38 MAPK pathway is selected from the group comprising dlk-1, mkk-4, and pmk-3.

6. A method of regenerating axons, the method comprising inhibiting a gene in the p38 MAPK pathway of C. elegans.

7. A method of regenerating axons in a subject, the method comprising inhibiting a gene with 80% or greater homology to a gene in the p38 MAPK pathway of C. elegans.

8. The method of claim 6 or 7, wherein the subject is a mammal.

9. The method of claim 8, wherein the mammal is a human.

10. The method of claim 6 or 7, wherein the gene in the p38 MAPK pathway is selected from the group comprising rpm-1 and fsn-1.

11. The method of claim 7, wherein the gene is inhibited with siRNA.

12. The method of claim 11, wherein the gene that is inhibited is phr-1.

13. A method of treating a subject with a neurodegenerative disease, the method comprising activating a gene with 80% or greater homology to a gene in the p38 MAPK pathway of C. elegans.

14. A method of treating a subject with a neurodegenerative disease, the method comprising inhibiting a gene with 80% or greater homology to a gene in the p38 MAPK pathway of C. elegans.

15. A method of regenerating axons in a subject, the method comprising activating a gene with 80% or greater homology to a gene in the p38 MAPK pathway of C. elegans by contacting the gene with a compound that activates said gene.

16. A pharmaceutical composition comprising the compound of claim 15.

17. A method of regenerating axons in a subject, the method comprising inhibiting a gene with 80% or greater homology to a gene in the p38 MAPK pathway of C. elegans by contacting the gene with a compound that inhibits said gene.

18. A pharmaceutical composition comprising the compound of claim 17.

19. A method of screening for a test compound that modulates the p38 MAPK pathway of C. elegans, comprising: (a) contacting a gene in the p38 MAPK pathway with a test compound; and (b) detecting interaction between the gene and the test compound, wherein interaction between the gene and the test compound indicates a test compound that modulates the p38 MAPK pathway.

20. The method of claim 19, wherein modulation further comprises activation of a gene in the p38 MAPK pathway.

21. The method of claim 19, wherein modulation further comprises activation of a gene with 80% or greater homology to a gene in the p38 MAPK pathway.

22. The method of claim 20, wherein the ability to modulate the p38 MAPK pathway is measured by contacting the test compound with one or more axons.

23. The method of claim 20, wherein the method comprises screening for a test compound that modulates the gene dlk-1 in the p38 MAPK pathway.

24. The method of claim 20, wherein the method comprises screening for a test compound that modulates the gene mkk-4 in the p38 MAPK pathway.

25. The method of claim 20, wherein the method comprises screening for a test compound that modulates the gene pmk-3 in the p38 MAPK pathway.

26. The method of claim 19, wherein a plurality of test compounds are contacted with a gene in the p38 MAPK pathway in a high throughput assay system.

27. The method of claim 26, wherein the high throughput assay system comprises an immobilized array of test compounds.

28. The method of claim 26, wherein the high throughput assay system comprises an immobilized array of a gene in the p38 MAPK pathway.

29. A compound identified by the method of claim 20.

30. The method of claim 19, wherein modulation further comprises inhibition of a gene in the p38 MAPK pathway.

31. The method of claim 19, wherein modulation further comprises inhibition of a gene with 80% or greater homology to a gene in the p38 MAPK pathway.

32. The method of claim 30, wherein the ability to modulate the p38 MAPK pathway is measured by contacting the test compound with one or more axons.

33. The method of claim 30, wherein the method comprises screening for a test compound that modulates the gene rmp-1 in the p38 MAPK pathway.

34. The method of claim 30, wherein the method comprises screening for a test compound that modulates the gene fsn-1 in the p38 MAPK pathway.

35. The method of claim 30, wherein the method comprises screening for a test compound that modulates the gene phr-1 in the p38 MAPK pathway.

36. The method of claim 30, wherein a plurality of test compounds are contacted with a gene in the p38 MAPK pathway in a high throughput assay system.

37. The method of claim 36, wherein the high throughput assay system comprises an immobilized array of test compounds.

38. The method of claim 37, wherein the high throughput assay system comprises an immobilized array of a gene in the p38 MAPK pathway.

39. A compound identified by the method of claim 30.
